(19) United States
(12) Patent Application Publication
(10) Pub. No.: US 2023/0329909 A1
(43) Pub. Date: Oct. 19, 2023

(54) SYSTEMS AND METHODS FOR DETERMINING THE CHARACTERISTICS OF STRUCTURES OF THE EYE INCLUDING SHAPE AND POSITIONS

(71) Applicant: Lensar, Inc., Orlando, FL (US)

(72) Inventors: Dustin Morely, Rockledge, FL (US); Gary P. Gray, Lake Mary, FL (US)

(73) Assignee: Lensar, Inc., Orlando, FL (US)

(21) Appl. No.: 18/123,245

(22) Filed: Mar. 17, 2023

Related U.S. Application Data
(60) Provisional application No. 63/320,870, filed on Mar. 17, 2022.

(51) Int. Cl. A61F 9/008; A61B 90/25; A61B 90/50; G16H 50/20; G16H 20/40; G06T 7/00

(52) U.S. Cl. CPC A61F 9/008; A61B 90/25; A61B 90/50; G16H 50/20; G16H 20/40; G06T 7/0016; A61B 2018/00994

(57) ABSTRACT

Systems, devices and methods for performing deep learning process to determine the characteristics of structures of the eye. Deep leaning, imaging devices and methods for laser and phacoemulsification operations. An integrated imaging device, laser-ultrasound, including femto-phaco, system and a computer vision device. Methods of training and using computer vision devices in ophthalmic treatment systems and therapies.

The present application claims priority and claims under 35 U.S.C. § 119(e)(1) the benefit of the filing date of U.S. provisional application serial No. 63/320,870 filed Mar. 17, 2022, the entire disclosure of which is incorporated herein by reference.

### BACKGROUND OF THE INVENTION

### Field of the Invention

The present inventions relate to devices, systems and methods for determining the characteristics of structures, including the shape and position of structures of the eye, using deep learning systems and methods that provide these characteristics from observed data and information. The present inventions further relate to integrated devices, systems and methods that used these deep learning systems and methods to perform highly accurate ophthalmic operations, such as therapeutic laser treatments, diagnostics, guided treatments, laser processing of materials, phacoemulsification and other ophthalmic related activities.

As used herein, unless specified otherwise, the terms “observed data”, “observed information”, “actual image”, “raw data”, “raw information”, and similar such terms are to be given their broadest possible meaning and would include information obtained from direct and indirect observation, monitoring, measuring, sensing and combinations and variations of these. Observed data would include, for example: data and information (a digital image, e.g., photograph, would include data and information) from direct observations, such as images from a camera, visual observation, optical coherence topography (OCT), images from Scheimpflug optical configurations, laser radar, ultrasound, MRI, x-ray, and other imaging and observational technologies, such as for example seismic and satellite. Observed data and information may be in compilations of data, which may further be sorted, indexed, tagged or otherwise categorized.

As used herein, unless specified otherwise, the terms “derived data”, “derived information” and similar such terms are to be given their broadest possible meaning and would include any data or information that results from or is obtained from performing an operation or calculation on data or information, including raw data and modeled data. For example, a photo editing application, such as Photoshop® would provide an edited image that is derived data. Accordingly, values such as averages are considered derived data, because they are derived from one or more operations on raw data. Although examples of simple (one, two or three) operations are provided above, it should be understood that tens, hundreds, thousands, and hundreds of thousands of operations or calculations, or more, may be performed on data to obtain derived data.

When derived data is stored, it becomes historic data, but also remains derived data, i.e., historic derived data. Derived data can be subjected to operations and calculations with the resulting information being derived data. Further, derived data, for example from observed data, can be combined with historic data, observed, modeled or derived, and used in operations and calculations to render additional derived data.

As used herein, unless specified otherwise, the terms “modeled data” and “modeled information” and similar such terms are to be given their broadest possible meaning and would include any data or information that is obtained from the theoretical construction or mathematical modeling of the characteristics of naturally occurring structures. An example of modeled data and information is anterior and posterior curvatures can be based on Kuszak aged lens models and Burd's numeric modeling, Burd et al. Vision Research 42 (2002) 2235-2251. The Burd model provides the following algorithm for anterior and/or posterior shape:

Z=aR5+bR4+cR3+dR2+f

The coefficients for this algorithm are set forth in Table 1, and the variables Z and R are defined by the drawing FIG. 9.

TABLE I

| | a | b | c | d | f |
|---|---|---|---|---|---|
| Anterior (11-year) | −0.00048433393427 | 0.00528772036011 | −0.01383693844808 | −0.07352941176471 | 2.18 |
| Posterior (11-year) | 0.00300182571400 | −0.02576464843559 | 0.06916082660799 | 0.08928571428571 | −2.13 |
| Anterior (29-year) | −0.00153004454939 | 0.01191111565048 | −0.02032562095557 | −0.07692307692308 | 2.04 |
| Posterior (29-year) | 0.00375558685672 | −0.03036516318799 | 0.06955483582257 | 0.09433962264151 | −2.09 |
| Anterior (45-year) | −0.00026524088453 | 0.00449862869630 | −0.01657250977510 | −0.06578947368421 | 2.42 |
| Posterior (45-year) | 0.00266482873720 | −0.02666997217562 | 0.08467905191557 | 0.06172839506173 | −2.42 |

Models can include the location of structures including the lens, the astigmatic axis, and any cataracts that may be present. Modeled data and information do not contain observed data, but can be used and combined with observed data to provide derived data.

As used herein, unless specified otherwise, the terms “machine learning model”, “deep learning model”, and similar such terms, are to be given their broadest possible meaning and would include any complete parameter set required to execute a given algorithm whereby such parameters were obtained by applying machine learning algorithms to a set of observed and/or derived data. Examples include deep neural networks, convolutional or otherwise.

As used herein, unless specified otherwise, the terms “characteristic”, “characterize”, and “characterization”, and similar such terms refers to information and data about the shape, location, composition, physical properties or position of a structure. Characteristics would include, for example, the location of a structure with respect to a fixed point, the location of a structure with respect to a coordinate system, the location of a structure with respect to a fix point in a coordinate system, the orientation of the structure (e.g., vertical axis, horizontal axis, equatorial axis, astigmatic axis, tilt, cyclotorsion), topography, optical properties (e.g., opacity, reflectivity, transmissivity), boundary layers, boundaries, cavities within the structure, roughness, compositional changes within the structure, and combinations and variations of these.

As used herein, unless specified otherwise, the term “determined characteristic” and similar such terms refers to the characteristics that are obtained from using the deep learning models and teachings of the present specification to processes observed data, raw data, derived data, historic data, modeled data and combinations and variations of these.

As used herein, unless specified otherwise, the term “quality” and similar such terms, when used with respect to derived data, augmented data, determined characteristics, and the characteristics of a structure, refers to the nature of such data and information, in particular, when such data and information is being used as basis for a procedure on, or related to, a structure. Thus, quality would refer to parameters such as resolution, accuracy, or both, as well as tolerances, clarity and reliability. Further, “higher quality” would indicate that, for example, an imaged had greater resolution, a location was more accurate or its accuracy increased, or its tolerances were smaller or narrower, and combinations of one or more of these.

The anatomical structures of the natural human eye are shown in general in FIG. 11, which is a cross sectional view of the eye. The sclera 131 is the white tissue that surrounds the lens 103 except at the cornea 101. The cornea 101 is the transparent tissue that comprises the exterior surface of the eye through which light first enters the eye. The iris 102 is a colored, contractible membrane that controls the amount of light entering the eye by changing the size of the circular aperture at its center (the pupil). The ocular or natural crystalline lens 103, a more detailed picture of which is shown in FIGS. 11A, (utilizing similar reference numbers for similar structures) is located just posterior to the iris 102. The terms ocular lens, natural crystalline lens, natural lens, natural human crystalline lens, and lens (when referring to the prior terms) are used interchangeably herein and refer to the same anatomical structure of the human eye.

Generally, the ocular lens changes shape through the action of the ciliary muscle 108 to allow for focusing of a visual image. A neural feedback mechanism from the brain allows the ciliary muscle 108, acting through the attachment of the zonules 111, to change the shape of the ocular lens. Generally, sight occurs when light enters the eye through the cornea 101 and pupil, then proceeds through the ocular lens 103 through the vitreous 110 along the visual axis 104, strikes the retina 105 at the back of the eye, forming an image at the macula 106 that is transferred by the optic nerve 107 to the brain. The space between the cornea 101 and the retina 105 is filled with a liquid called the aqueous 117 in the anterior chamber 109 and the vitreous 110, a gel-like clear substance, in the chamber posterior to the lens.

FIG. 11A illustrates, in general, components of and related to the lens 103 for a typical 50-year old individual. The lens 103 is a multi-structural system. The lens 103 structure includes a cortex 113, and a nucleus 129, and a lens capsule 114. The capsule 114 is an outer membrane that envelopes the other interior structures of the lens. The lens epithelium 123 forms at the lens equatorial 121 generating ribbon-like cells or fibrils that grow anteriorly and posteriorly around the ocular lens. The nucleus 129 is formed from successive additions of the cortex 113 to the nuclear regions. The continuum of layers in the lens, including the nucleus 129, can be characterized into several layers, nuclei or nuclear regions. These layers include an embryonic nucleus 122, a fetal nucleus 130, both of which develop in the womb, an infantile nucleus 124, which develops from birth through four years for an average of about three years, an adolescent nucleus 126, which develops from about four years until puberty which averages about 12 years, and the adult nucleus 128, which develops at about 18 years and beyond.

The embryonic nucleus 122 is about 0.5 mm in equatorial diameter (width) and 0.425 mm in Anterior-Posterior axis 104 (AP axis) diameter (thickness). The fetal nucleus 130 is about 6.0 mm in equatorial diameter and 3.0 mm in AP axis 104 diameter. The infantile nucleus 124 is about 7.2 mm in equatorial diameter and 3.6 mm in AP axis 104 diameter. The adolescent nucleus 126 is about 9.0 mm in equatorial diameter and 4.5 mm in

AP axis 104 diameter. The adult nucleus 128 at about age 36 is about 9.6 mm in equatorial diameter and 4.8 mm in AP axis 104 diameter. These are all average values for a typical adult human lens approximately age 50 in the accommodated state, ex vivo. Thus this lens (nucleus and cortex) is about 9.8 mm in equatorial diameter and 4.9 mm in AP axis 104 diameter. Thus, the structure of the lens is layered or nested, with the oldest layers and oldest cells towards the center.

The lens is a biconvex shape as shown in FIGS. 11 and 11A. The anterior and posterior sides of the lens have different curvatures and the cortex and the different nuclei in general follow those curvatures. Thus, the lens can be viewed as essentially a stratified structure that is asymmetrical along the equatorial axis and consisting of long crescent fiber cells arranged end to end to form essentially concentric or nested shells. The ends of these cells align to form suture lines in the central and paracentral areas both anteriorly and posteriorly. The older tissue in both the cortex and nucleus has reduced cellular function, having lost their cell nuclei and other organelles several months after cell formation.

As used herein unless specified otherwise, the recitation of ranges of values herein is merely intended to serve as a shorthand method of referring individually to each separate value falling within the range. Unless otherwise indicated herein, each individual value within a range is incorporated into the specification as if it were individually recited herein.

Generally, the term “about” as used herein unless stated otherwise is meant to encompass the larger of a variance or range of ±10% and the experimental or instrument error associated with obtaining the stated value.

As used herein, unless specifically stated otherwise, the terms “phacoemulsification”, “phaco”, “phaco system”, are to be given their broadest construction possible, refer to the same general equipment and procedures and generally relate to the use of ultrasonic energy to drive a needle or tip to, for example, cut, fragment, separate, and emulsify tissue, including tissue of the eye, such as the lens and cataracts. Such procedures and systems may also include components and methods for aspiration, irrigation and both.

As used herein, unless specifically stated otherwise, the terms “femtosecond laser,” “femtosecond laser beam”, “femtosecond pulse”, and similar such terms, are used to refer to the pulse duration, and thus also pulse length of a laser beam (which can also be referred to as pulse width), and would mean all lasers and laser beams with pulse durations of less than 1 picosecond (less than 1×10⁻¹² seconds) to and including 1 femtosecond (fs) (1×10⁻¹⁵ seconds).

As used herein, unless specifically stated otherwise, the terms “picosecond laser,” “picosecond laser beam”, “picosecond pulse”, and similar such terms, are used to refer to the pulse duration, and thus also pulse length of a laser beam, (which can also be referred to as pulse width) and would mean all lasers and laser beams with pulse durations of 1 picosecond (ps) (1×10⁻¹² seconds) up to 1 nanosecond (ns) (1×10⁻⁹ seconds).

As used herein, unless specifically stated otherwise, the terms “distal” and “proximal” have the following means. For the laser, laser beam, and laser components, distal means the side, location or position that is closer to the laser beam source. For the phacoemulsification system, distal means the side, location or position, that is closer to the ultrasound energy source. For the laser, laser beam and laser components, the term proximal means the side, location or position that is further away from, along the laser beam path, the laser beam source; and thus, in operation closer to the patient. For the phacoemulsification system, the term proximal means the side, location or position that is further away from, along the energy transmission path, the ultrasound energy source; and thus, in operation closer to the patient. Conversely, the distal end of the laser component or the phacoemulsification component are further from the patient during operation of those systems.

Ultrasonic energy, in addition to being a diagnostic tool, has therapeutic uses. Ultrasonic energy can be focused, directed, used to move, e.g., ocellate or vibrate, cutting devices, tools or tips to cut, soften or emulsify tissue, to create mists and vapors, and combinations and variations of these. In general, phacoemulsification is a medically recognized technique that uses ultrasonic energy for crystalline lens removal. Generally, phacoemulsification includes making a corneal incision, a scleral incision and one or more and both of these. The insertion through one of those incisions of a phacoemulsification handpiece, which is typically comprised of a needle that is ultrasonically driven, in order to, for example, emulsify (i.e., to liquefy), the natural crystalline lens, break a cataract into small pieces, and combinations and variations of these. The emulsified pieces may subsequently be removed using the same handpiece or another handpiece. The surgeon may then insert implants in the eye through the incision.

In general, therapeutic laser procedures for the eye involve positioning the patient on a bed, or patient support, aligning the eye with the laser beam path of the laser system and attaching a patient interface between the laser system and the eye. The therapeutic laser beam is then delivered in a laser beam pattern to perform a therapeutic laser operation on the eye, and in particular, the structures of, or associated with the eye to address conditions of the eye. Thus, for example, laser procedures, to address cataracts, presbyopia, refractive errors (both natural and induced) and other conditions of the eye that are known to the art.

This Background of the Invention section is intended to introduce various aspects of the art, which may be associated with embodiments of the present inventions. Thus, the forgoing discussion in this section provides a framework for better understanding the present inventions, and is not, and should not be viewed as, an admission of prior art.

### SUMMARY

There has existed a long standing and unfulfilled need to address and improve, systems and methods that characterize structures, the quality of the characterizations of structures obtained from these systems and methods, and in particular, the characterization, and the quality of the characterization, of structures of the eye. There further exists a long standing and unfulfilled need to address and improve the ability to automatically direct and target laser beams, and other energy forms, to a specific structure or location in or on that structure for use in activities, including therapeutic operations. Among these activities, where this long standing and unfilled need is present, is in the field of surgery and targeted energy therapies. These long standing needs have been present and continue, in among other things, the field of ophthalmology, including addressing cataracts, addressing refractive based issues, addressing presbyopia, addressing diseases, conditions and injuries of the eye, as well as, in other procedures on, and conditions of, the eye and nearby structures. The present inventions, among other things, solve these and other needs by providing the articles of manufacture, devices and processes set forth in this specification, drawings and claims.

Thus, there is provided an ophthalmic therapeutic laser system, having: an assembly, the assembly comprising: a therapeutic laser for providing a therapeutic laser beam along a laser beam path; an arm attached to the assembly; the arm having a distal end and a proximal end, wherein the distal end is attached to the assembly; wherein the proximal end has a laser delivery head; wherein the arm contains a portion of the laser beam delivery path; and, a deep learning means for providing one or more of an image, data and information for a structure of an eye.

Further, there is provided an ophthalmic therapeutic laser system, comprising: an assembly, the assembly comprising: a therapeutic laser for providing a therapeutic laser beam, in a laser beam pattern, along a laser beam path; an arm attached to the assembly; the arm having a distal end and a proximal end, wherein the distal end is attached to the assembly; wherein the proximal end has a laser delivery head; wherein the arm contains a portion of the laser beam delivery path; and, a deep learning means for providing a determined characteristic about a structure of an eye.

Moreover, there is provided a method of adjusting the delivery of an ophthalmic therapeutic laser beam pattern, in an ophthalmic therapeutic laser beam system, the method including: obtaining raw image data from an iris of an eye; processing the raw image data in a deep learning means to thereby provide a determined characteristic of the eye; using the determined characteristic to adjust a delivery location for the therapeutic laser beam pattern.

There is further provided these systems and methods having one or more of the following features: wherein the deep learning means provides targeting information for the direction, placement or both of a therapeutic laser beam shot pattern; wherein the targeting information comprises a cyclotorsion of the eye based solely upon a retina of the eye; wherein the system further comprises a phacoemulsification system for providing therapeutic ultrasonic energy to the eye; wherein the phacoemulsification system in integrated with the laser system and shares, at least a part of, one or more of a common housing, a common control system, a common power source; wherein the deep learning means comprises a computer vision device (CVD); wherein the deep learning means comprises a computer vision device (CVD), wherein the CVD is based upon a convolutional neural network; wherein the deep learning means comprises a computer vision device (CVD), wherein the CVD is trained by a convolutional neural network; wherein the deep learning means comprises a computer vision device (CVD), wherein the CVD is based upon a U-Net approach to information;

wherein the deep learning means comprises a computer vision device (CVD), wherein the CVD is trained by a U-Net approach to information. The laser system of any of claims 11 to 16, wherein the structure of the eye is the retina; wherein the determined characteristics are not based upon an individual markers or specific reference points on the iris; wherein the determined characteristic provides in part targeting information for the delivery of the laser beam pattern; wherein the determined characteristic is the cyclotorsion of the eye; wherein the determined characteristic is the cyclotorsion of an undilated pre-treatment eye and a dilated treatment eye; wherein the determined characteristic is the cyclotorsion of an eye having different amounts of dilation between pre-treatment and treatment; wherein the system further comprises a phacoemulsification system for providing therapeutic ultrasonic energy to the eye; and, wherein the system further comprises a phacoemulsification system for providing therapeutic ultrasonic energy to the eye; wherein the phacoemulsification system in integrated with the laser system and shares, at least a part of, one or more of a common housing, a common control system, a common power source.

In addition, there is provided any of these systems or methods wherein: the integration control system, the therapeutic laser control system or both have a plurality of predetermined laser delivery patterns; the integration control system, the phacoemulsification control system or both have, a plurality of predetermined phacoemulsification procedures; and, the deep learning means is configured to determine information about a cataract in a lens of an eye, and the system is configured based upon that determined information to recommend, at least in part, a laser-phaco combined therapy based upon the determined information about the cataract; wherein the laser-phaco combined therapy comprises: at least one of the plurality of predetermined laser delivery patterns; and, at least one of the plurality of predetermined phacoemulsification producers.

Further, there is provided a method of using an integrated laser-phaco system including: a GUI; a therapeutic laser for providing a therapeutic laser beam along a laser beam delivery path, comprising a therapeutic laser control system; a phacoemulsification system for providing therapeutic ultrasonic energy, comprising a phacoemulsification system control system; an integration control system in control communication with the therapeutic laser control system, a deep learning means for providing an image, data and/or information for a structure of an eye, the phacoemulsification system and the GUI, to determine, provide or both, a laser-phaco combined therapy for a cataractous eye of a patient, the method including: the system evaluating information about a cataract in a lens of the cataractous eye of the patient; the system determining a recommended laser-phaco combined therapy, based at least in part, upon the determined information about the cataract; wherein the recommended laser-phaco combined therapy comprises a predetermined laser delivery pattern, and a predetermined phacoemulsification procedure; the system displaying on the GUI the menu items relating to the recommended laser-phaco combined therapy; the system receiving a selection of the recommended laser-phaco combined therapy for deliver to the lens of the eye of the patient.

Still further there is provided a system having: an ophthalmic device; and, a deep learning means for providing a determined characteristic of an eye.

There is further provided these systems and methods having one or more of the following features: wherein the determined characteristic is one or more of an image, data, targeting information for a structure of an eye; and, wherein the structure of the eye is an iris.

In addition, there is provided a laser-ultrasound system, having an imaging device equipped with one or more machine learning models, the system including: an assembly, the assembly including: a therapeutic laser for providing a therapeutic laser beam along a laser beam path; a phacoemulsification system for providing therapeutic ultrasonic energy; an arm attached to the assembly; the arm having a distal end and a proximal end, wherein the distal end is attached to the assembly; wherein the proximal end has a laser delivery head; wherein the arm contains a portion of the laser beam delivery path; wherein the assembly is configured to be located at an angle with respect to a patient position.

Yet further, there is provided a laser-ultrasound system having an imaging device equipped with one or more machine learning models, the system including: a therapeutic laser for providing a therapeutic laser beam along a laser beam path; a phacoemulsification system for providing therapeutic ultrasonic energy; and optics defining four pupils in the system, and wherein the laser beam path extends through at least two of the pupils.

Moreover, there is provided a laser-ultrasound system, having an imaging device equipped with one or more machine learning models, the system including: a therapeutic laser system; a phacoemulsification system for providing therapeutic ultrasonic energy; and, a safety interlock, whereby the laser system is prevented from firing the laser when the phacoemulsification system is in operation.

Furthermore, there is provided a laser-ultrasound system having an imaging device equipped with one or more machine learning models, the system including: a means for providing a first and a second therapeutic laser beam; the system having optics defining a laser beam path; the first and the second laser beam path traveling along the laser beam path; wherein the first therapeutic laser beam has a pulse width of about 1,000 fs to about 2000 fs; the system including a laser beam delivery pattern for performing a lens cut with the first therapeutic laser beam; wherein the second therapeutic laser beam has a pulse width of about 100 fs to about 500 fs; the system including a laser beam delivery pattern for performing a corneal cut with the second therapeutic laser beam; and, a phacoemulsification system for providing therapeutic ultrasonic energy.

Moreover, there is provided, these laser systems, methods and devices having one or more of the following features: wherein the wavelength of the first laser beam is 1030 nm; wherein the wavelength of the second laser beam is 1030 nm; wherein the wavelength of the first laser beam and the second laser beam are the same; wherein the repetition rate the firs laser beam, the second laser beam or both, is 320 kHz or less; wherein the system comprises a surgical microscope; and the surgical microscope is integral with the system and configured to receive one or more of images, data, information from the laser system; wherein the surgical microscope is configured to display the received images, data or information during a laser procedure, a phacoemulsification producer, or both; wherein the system comprises a 3D viewing system; and the 3D viewing system is integral with the system and configured to receive one or more of images, data, information from the laser system; and, wherein the 3D viewing system is configured to display the received images, data or information during a laser procedure, a phacoemulsification producer, or both.

Still additionally there is provided an integrated laser-ultrasound system, having an imaging device equipped with one or more machine learning models, the system including: a first housing, a second housing, a GUI and a means for optically connecting the first housing and the second housing; wherein the second housing is moveably associated with the first housing; an assembly, the assembly including: a therapeutic laser for providing a therapeutic laser beam along a laser beam delivery path, including a therapeutic laser control system; a phacoemulsification system for providing therapeutic ultrasonic energy, including a phacoemulsification system control system; wherein at least a portion of the therapeutic laser and the phacoemulsification system are located within the first housing; an integration control system in control communication with the imaging device, the system therapeutic laser control system, the phacoemulsification system and the GUI.

Moreover, there is provided, these laser systems having an imaging device equipped with one or more machine learning models, the system including a foot switch in control communication with at least one or more of: the phacoemulsification system control system; the imaging device, the therapeutic laser control system; and the integration control system.

Moreover, there is provided, these laser systems, methods and devices having one or more of the following features: wherein the system, and in embodiments the imaging device, determines the information about the cataract in the lens of the cataractous eye of the patient based in whole or in part upon the application of one or more machine learning models to the imaging device output.

Furthermore, there is provided, these laser systems, methods and devices having one or more of the following features: the system having an iris registration system that interoperates with the imaging devices and machine learning models.

Moreover, there is provided, these laser systems, methods and devices having one or more of the following features: wherein the therapeutic laser is selected from the group consisting of a femto second laser and a pico second laser; wherein the system is non-handed; wherein the system comprises a phaco tray, a phaco cassette and is non-handed.

Moreover, there is provide the method of servicing, upgrading a software, operating, or preforming a surgery using, any of these systems.

### BRIEF DESCRIPTION OF THE DRAWINGS

FIG. 1A is an image of the eye, in accordance with the present inventions.

FIG. 1B is a table showing an embodiment of the data of the image of the eye of FIG. 1A, in accordance with the present inventions.

FIGS. 3, and 3A to 3I are tables illustrating an embodiment of the steps in a convolution approach for data processing, in accordance with the present inventions.

FIG. 4 is an illustration of an embodiment of a convolutional neural network deep learning model, in accordance with the present inventions

FIG. 5 is an illustration of an embodiment of a U-Net approach to information hierarchy, in accordance with the present inventions.

FIG. 6 is a chart showing an embodiment of a momentum process or tool, in accordance with the present inventions.

FIGS. 7A and 7B are embodiments of images of the eye for training and validation purposes of an embodiment of a device for providing determined characteristics of an eye, such as a convolutional neural network (CCN), in accordance with the present inventions.

FIG. 8 are embodiments of images of the eye for training and validation purposes of an embodiment of a device for providing determined characteristics of an eye, such as a convolutional neural network (CCN), in accordance with the present inventions.

FIG. 9 is a cross section schematic of an eye showing the variables of the Burd model algorithm, for use in accordance with the present inventions.

FIG. 10 is a graph showing an embodiment of training and validation losses, as losses vs iteration training, in accordance with the present inventions.

FIGS. 11 and 11A are cross sections of the eye.

FIG. 12 is a schematic of an embodiment of an ophthalmic therapeutic laser system in accordance with the present inventions.

FIG. 13 is a perspective view of an embodiment of a femto-phaco system, in accordance with the present inventions.

FIG. 14 is a perspective view of an embodiment of a femto-phaco system, in accordance with the present inventions.

### DESCRIPTION OF THE PREFERRED EMBODIMENTS

In general, embodiments of the present inventions are devices, systems and methods for determining the characteristics of structures. These determined characteristics are provided using deep learning methods and devices, based upon data and information obtained by or provided to the devices, such as observed data and information, historic data and information, and modeled data and information.

In general, these determined characteristics can have better quality (e.g., resolution, accuracy, or both) than the data and information upon which they are based. In this manner, embodiments of the present invention provide determined characteristics that can be far better quality than the observed data and information upon which they are based, the modeled data and information upon which they are based, or both, as well as other data and information, such as historic data upon which they may be based. Thus, for example, the determined characteristics can provide a more accurate shape, position and location of a structure, e.g., lens capsule, pupil, sclera, the iris, than is provided by the observed data alone. In preferred embodiments of the present inventions, the determined characteristics can be of such high quality, that cannot be obtainable from the observed data by means other than the present inventions. In embodiment there is an improvement in the success rate (i.e. decreases the odds of having to skip part or all of the treatment due to being unable to find some structure, or otherwise having a system determine that there is insufficient data to proceed safely). In a preferred embodiment, the embodiment's classification of individual image pixels is of far higher quality than the steps or methods of prior systems, e.g., edge detection.

In general, the determined characteristics of the structures, can include, among other things, the shape, the position or location (with respect to a fixed point, a coordinate system, or both), the orientation (e.g., astigmatic axis, tilt, cyclotorsion), topography, optical properties (e.g., opacity, reflectivity, transmissivity), boundary layers, cavities within the structure, compositional changes within the structure, and combinations and variations of these.

In general, embodiments of these devices, systems and methods find application in, and are configured to, determine the characteristics of the structures of the human body, such as organs, the circulatory system, and in particular the eye (animal, mammal and human) and its structures (e.g., lens, lens capsule, iris, sclera, pupil, cataractous tissue, corneal surfaces).

Embodiments of the present inventions further relate to integrated devices, systems and methods that used these deep learning methods and devices to perform highly accurate operations, such as therapeutic laser treatments, diagnostics, guided treatments, laser processing of materials and other activities. These integrated systems, methods and devices would include, for example, combinations of medical, e.g., therapeutic, diagnostic or both, devices with embodiments of the present deep learning characterization methods and devices. Thus, these integrated medical systems, methods and devices would include, embodiments of the present deep learning characterization devices and methods combined with, for example, ultrasound imaging devices, laser imaging devices, ultrasound therapeutic devices, MRI, and laser therapeutic systems and methods.

Embodiments of the present integrated medical systems, methods and device include integrated ophthalmic devices, systems and methods, which would include, embodiments of the present deep learning characterization devices and methods combined with, for example, therapeutic laser devices, diagnostic laser devices, and therapeutic-diagnostic laser devices, laser eye surgery devices, Optical Coherence Tomography (OCT) devices, optical diagnostic systems, phacoemulsification devices, and combinations and variations of these. Thus, these integrated ophthalmic systems, methods and devices, have an ophthalmic diagnostic, therapeutic or both system and a deep learning characterization system. As such, these integrated ophthalmic devices can also be referred to as integrated deep learning ophthalmic systems, and similar such terms.

In a preferred embodiment of the integrated deep learning ophthalmic systems, there is provided an embodiment of the present deep learning characterization devices and methods combined with a laser-phaco systems (i.e., an integrated system having both a therapeutic laser and a phacoemulsification system), such as disclosed in US Pat. Publ. No. 2021/0259880, the entire disclosure of which is incorporated herein by reference.

In general, these integrated deep learning ophthalmic devices, methods and systems, are configured to, and capable of addressing various conditions of the eye, and performing various procedures including: capsulotomies; custom shaped non-circular and non-elliptical capsulotomies: lens cutting, fragmentation, sectioning and removal; cataract cutting, fragmentation, separation and removal; emulsification of lens and cataract tissue; corneal cutting and incisions; creation of corneal flaps and pockets; making limbal relaxing incisions (LRI); addressing and correcting refractive errors (natural and induced): removal of residual cortical material; removal of lens epithelial cells; vitreous aspiration and cutting associated with anterior vitrectomy; addressing bipolar coagulation; and intraocular lens injection; keratoplasty, radial keratotomy (RK)(e.g., micro-RK, micro-RK/AK and traditional RK); astigmatic keratotomy (AK) and combinations and variations of these, as well as, retinal repairs and diagnosis; photocoagulation; glaucoma therapies.

Preferred embodiments of the present deep learning characterization devices and methods provide high quality characteristics of structures from observed data and information that is limited (e.g., limited number of data points, limited distribution of data points, limited area(s) of data points), less than optimal, poor quality, low resolution, and combinations of these and other deficiencies. Thus, by way of illustration, the present deep learning characterization methods and devices can obtain highly accurate shape and position characterization of the structures of an eye, even under challenging circumstances such as partial occlusion of structures or anomalous anatomical conditions. In this manner, preferred embodiments of the present deep learning characterization devices and methods provide characterizations that have the quality (e.g., accuracy, resolution, clarity) necessary to direct a therapeutic procedure from observed data and information. In this manner, embodiments of the present inventions, permit the safe and efficacious use of observed data and information, that otherwise would be of low quality, low reliability, limited data, not directly observable, usable and combinations and variations of these.

### Therapeutic Laser and System—Generally

Any laser that is configured to prove a laser beam that is useful, safe and effective for treating the eye, its structures and adjacent tissues, and conditions thereof, can be used to provide a therapeutic laser beam. Tunable lasers, adjustable lasers and combinations and variations of these lasers, can be used, e.g., pulse width can be varied, pulse rate can be varied, power can be varied and wavelength can be varied. More than one therapeutic laser can be used. The therapeutic laser can be a pulsed laser, such as femto second laser or a pico second laser, and longer and shorter pulses, a continuous laser and combinations of these.

The therapeutic laser can have a wavelength in the IR spectrum, the UV spectrum, as well as other wavelengths. The therapeutic laser beams can have wavelengths of from about 300 nm to about 2,500 nm, from about 1,000 nanometers (nm) to about 1,300 nm, 1020 nm, about 1020 nm, 1030 nm, about 1030 nm, 1040 nm, about 1040 nm, 1050 nm, about 1050 nm, and from about 1020 to about 1050 nm, and combinations and variations of these as well as other wavelengths.

The therapeutic laser can have pulse durations of from about 1 fs to about 100 ps, from about 200 fs to about 500 ps, from about 300 fs to about 100 ps, from about 300 fs to about 10 ps, from about 300 fs to about 2,000 fs, and combinations and variation of these wavelengths, as well as longer and shorter durations. The system can have one or more “short pulse duration” therapeutic lasers having pulse widths of 300 fs, about 300 fs, 350 fs, about 350 fs, 400 fs, about 450 fs, 500 fs, about 500 fs, from about 300 fs to about 600 fs and combinations and variations of these. The system can have one or more “long pulse duration” therapeutic lasers having pulse durations of 1000 fs, about 1000 fs, 1200 fs, about 1,200 fs, 1,300 fs, about 1,300 fs, 1,500 fs, about 1,500 fs, from about 1,200 fs to about 1,600 fs and combinations and variations of these.

These dual beam embodiments, i.e., having at least both short pulse duration and long pulse duration therapeutic laser beams have several advantages and benefits that would include, for example, the ability to reduce the need and use of equipment such as diamond blades and knifes. Thus, reducing costs, risks of infection and the time of the procedure.

The therapeutic laser beams can have pulse repetition rates of from about 50 kilohertz (kHz) to about 5 megahertz (Mhz), from about 50 kHz to bout 2 Mhz, from about 50 kHz to about 1 Mhz, from about 50 kHz to about 750 kHz, from about 100 kHz to about 200 kHz, from about 150 kHz to about 350 kHz, about 100 kHz, about 150 kHz, about 200 kHz, about 300 kHz and variations and combinations of these and greater and smaller rates.

The therapeutic laser beams can average output power at a specified pulse repetition rate of from about 1 Watt (W) to about 8 W, from about 2.5 W to about 5 W, from about 3 W to about 4.5 W, from 3 W to 5 W, less than 6 W, less than 5 W, any power where laser induced optical breakdown (LIOB), photodisruption or both occurs and combinations and variations of these, and lower and higher powers.

Embodiments of these system also can perform sub-threshold treatments, diagnostics and combinations and variations of these. Thus, the therapeutic laser beam can be delivered to the eye at a power, or in a manner, that is below the point where LIOB would occur. The therapeutic laser beam can be delivered to the eye at a power or in a manner where the power is below the point where photodisruption occurs. Thus, in an embodiment of the present procedures, a sub-threshold laser procedure can be performed, a laser procedure that induces photodisruption can be performed and a phacoemulsification can be performed, and combinations and variations of some or all of these procedures, can be performed without moving the location of the patient or the device.

The therapeutic laser beams can have a pulse energy of from about 1 nanojoule (nJ) to about 2 millijoule (mJ), from about 1 nJ to about 1 mJ, from about 2 microjoules (μJ) to about 70 μJ, from about 5 μJ to about 45 μJ, from about 2 μJ to about 35 μJ, from about 10 μJ to about 30 μJ, less than 45 μJ, less than 35 μJ, any pulse energy where photodisruption, LIOB or both occurs, and combinations and variations of these, and lower and higher energies.

The therapeutic laser beams of the present systems can have one or more of the above beam features, e.g., wavelength, duration, repetition rate, power, and pulse energy, and combinations and variations of these.

A Yb:YAG laser that generates ultrashort laser pulses in the 1030 nm wavelength can be used as a therapeutic laser. In general, the therapeutic laser provides a beam that is of a wavelength that transmits through the cornea, aqueous and lens. The beam can be of a short pulse length, together with the energy and beam size, to produce photodisruption, LIOB or both of the targeted ocular tissue, e.g., cornea, limbus, lens capsule, lens, cataractous tissue, opacified tissue, and other tissue. Thus, as used herein, the term laser shot or shot refers to a laser beam pulse delivered to a location that by itself or in combination with other pulses results in a therapeutic effect, e.g., LIOB. As used herein, the term photodisruption essentially refers to the conversion of matter to a gas by the laser. In embodiments, wavelengths of about 300 nm to 2500 nm may be employed. Pulse widths from about 1 femtosecond to 100 picoseconds may be employed. Energies from about a 1 nanojoule to 1 millijoule may be employed. The pulse rate (also referred to as pulse repetition frequency (PRF) and pulses per second measured in Hertz) may be from about 1 kHz to several GHz. Generally, lower pulse rates correspond to higher pulse energy in commercial laser devices. A wide variety of laser types may be used to have a therapeutic effect, e.g., cause photodisruption, LIOB or both of ocular tissues, dependent upon pulse length and energy density, as well as other factors. Thus, examples of such lasers would include: the Delmar Photonics Inc. Trestles-20, which is a Titanium Sapphire (Ti:Sapphire) oscillator having a wavelength range of 780 to 840 nm, less than a 20 femtosecond pulse width, about 100 MHz PRF, with 2.5 nanojoules; the Clark CPA-2161, which is an amplified Ti:Sapphire having a wavelength of 775 nm, less than a 150 femtosecond pulse width, about 3 KHz PRF, with 850 microjoules; the IMRA FCPA (fiber chirped pulse amplification) μjewel D series D-400-HR, which is a Yb:fiber oscillator/amplifier having a wavelength of 1045 nm, less than a 1 picosecond pulse width, about 5 MHz PRF, with 100 nanojoules; the Coherent Staccato, which is a YB:Yag having a wavelength of 1030 nm, about 1.5 picosecond pulse width, about 80 KHz PRF, with 30 microjoules; and, the Coherent Rapid, which is a YB:Yag having a wavelength of 1030 nm, about 1.5 picosecond pulse width, and can include one or more amplifiers to achieve approximately 2.5 to 10 watts average power at a PRF of between 25 kHz to 650 kHz and also includes a multi-pulsing capability that can gate two separate 50 MHz pulse trains. and, the IMRA FCPA (fiber chirped pulse amplification) μJewel D series D-400-NC, which is a Yb:fiber oscillator/amplifier having a wavelength of 1045 nm, less than a 100 picosecond pulse width, about 200 KHz PRF, with 4 microjoules. These and other similar lasers may be used a therapeutic laser and to generate therapeutic laser beams.

Embodiments of laser systems, methods and apparatus for performing laser operations on the eye are disclosed and taught in US patent application Publication Nos. 2016/0302971, 2015/0105759, 2014/0378955, and U.S. Pat. Nos. 8,262,646 and 8,708,491, the entire disclosures of each of which are incorporated herein by reference.

### Laser Beam Delivery—Generally

In general, embodiments of the optics for delivering the therapeutic laser beam to the natural lens of the eye should be capable of providing a series of shots to the natural lens in a precise and predetermined pattern in the x, y and z dimension. The optics should also provide a predetermined beam spot size to cause photodisruption, LIOB or both with the laser energy reaching the natural lens or other targeted tissue. Thus, the optics may include, without limitation: an x y scanner; a z focusing device; and, focusing optics. The focusing optics may be conventional focusing optics, flat field optics, telocentric optics, and combinations and variations of these, each having corresponding computer controlled focusing, such that calibration in x, y, z dimensions is achieved. For example, an x y scanner may be a pair of closed loop galvanometers with position detector feedback. Examples of such x y scanners would be the Cambridge Technology Inc. Model 6450, the SCANLAB hurrySCAN and the ACRES Rhino Scanner. Examples of such z focusing devices would be the Phsyik International Peizo focus unit Model ESee Z focus control and the SCANLAB varrioSCAN.

### Laser Control System—Generally

In general, embodiments of the control system for delivering the therapeutic laser beam may be any computer, controller, software hardware and combinations and variations of these that is capable of selecting and controlling x y z scanning parameters and laser firing, among other things. These components may typically be associated at least in part with circuit boards that interface to the x y scanner, the z focusing device, the laser and combinations and variations of these. Among other things, the laser control system may contain the programs that direct the laser through one or more laser shot patterns. The laser control system also, has the further capabilities of integrating with the system control system, and function with, or otherwise working as an integrated system with the ultrasonic control, and the monitor or control panel. The system controller, the laser controller, the ultrasound controller and combinations and variations of these can also control other components of the system, as well as, maintaining data, data, analyzing data and images, preparing and suggesting table and treatments, and performing calculations. The control system may contain the programs that direct the laser through one or more laser shot patterns.

### Obtaining Observed Data and Information—Generally

Generally, observed data and information in the form of images (optical images) travel back from the structure of the eye, the PID, or other structures, to the device or system along an optical path, e.g., along an image path, which can be through free space, optical components (lens, mirrors, fibers etc.) and both.

In general, in embodiments, the assembly or device for obtaining observed data and information for the purpose of providing determined characteristic, such as the shape and position, of the eye and structures within the eye, can be an optical coherence tomography (OCT), a Scheimpflug device having a single moveable camera, multiple fixed cameras, combinations and variations of these, configurations of a light source(s) and camera(s), and other types of devices for obtaining an image, partial image, reflected or refracted light from the eye and structures within the eye.

Obtaining the observed data and information may be accomplished by several methods and apparatus. For example, x y centration of the lens may be accomplished by observing the lens through a co-bore sighted camera system and display. The z position observed data and information may be determined by a range measurement device utilizing optical triangulation or laser and ccd system, such as the Micro-Epsilon opto NCDT 1401 laser sensor, the Aculux Laser Ranger LR2-22 and combinations and variations of these. The use of a 3-dimensional viewing and measurement apparatus may also be used to determine observed data and information, such as an x, y and z positions of the lens. For example, the Hawk 3 axis non-contact measurement system from Vision Engineering could be used to obtain this observed data and information.

Yet a further example of an apparatus that can be used to obtain observed data and information would comprise a camera or cameras, which can view a reference and the natural lens, and would also include a light source to illuminate the natural lens. Such light source could be a structured light source, such as for example a slit illumination designed to generate point information, or a 3-dimensional information based upon geometry. Further one, two, three, four or more, light sources can be positioned around the eye and the electronically activated to provide multiple views, planar images, of the eye, and in particular the cornea and the lens, at multiple planar slices that can then be integrated to provide data for position and location information relative to the laser system about those structures.

Examples of assemblies, methods, devices that can be used for obtaining observed data and information for the eye and structures within the eye, are disclosed and taught in US patent publications and patents numbers 2018/0085256, 2016/0302971, 2015/0105759, 2012/0330290, 2016/0030244, U.S. Pat. Nos. 9,180,051 and 8,708,491, the entire disclosure of each of which is incorporated herein by reference. These assemblies, methods, devices also provide the ability to obtain modeled data and information, as well as, derived data and information for the eye and structures within the eye. Iris registration devices for determining derived data and information about the angular orientation of the eye are taught and disclosed in US patent publication number 2015/0105759, the entire disclosure of which is incorporated herein by reference.

### Providing Determined Characteristics—Generally

In general, imaging devices coupled with computer vision software provide the observed data and information, for example about the characteristics of the eye and structures of the eye. These composite devices contain a computer, generally having a processor, memory and programs comprising algorithms. These devices are configured for, and employ, machine learning, artificial intelligence and preferably deep learning to provide the determined characteristics of the structure, for example determined characteristics of the eye and structure within the eye. The determined characteristics provided by these devices can have substantially greater utility for diagnostics, therapeutics and both than the observed data and information upon which they were determined. In general, such devices and processes are referred to herein as computer vision devices and processes (CVD).

Thus, in embodiments, the output of such a computer vision system includes determined data and information, such as, the relative distance of portions of the lens, the shape of the lens, the pupil, or other structures of the eye or tissue adjacent to the eye, from a laser, for example the optics head. In embodiments, this distance is maintained constant by for example the PID. In embodiments, this device determines the position of the lens and other structures, with respect to the scanning coordinates for the laser delivery pattern in all three dimensions, in which case the device is providing augmented targeting information and data.

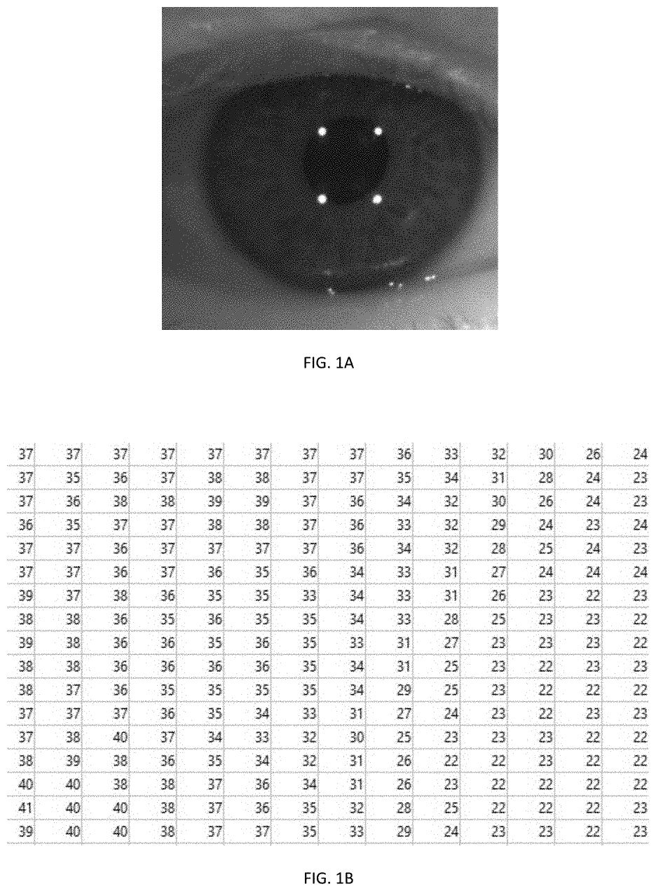

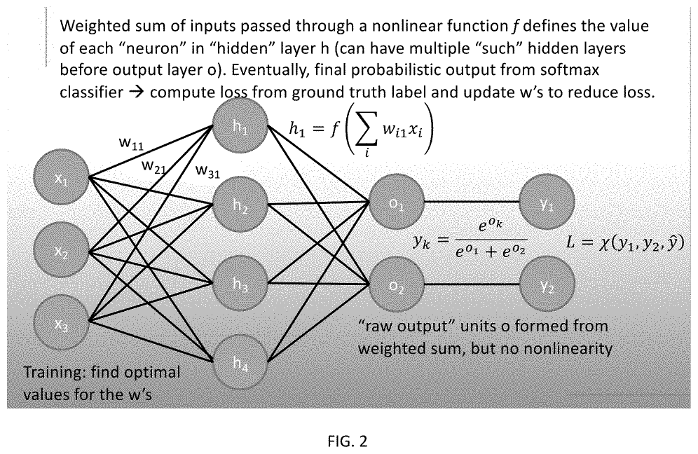

Generally, the difference between how observed information and data is perceived or understood, i.e., “seen” by human vision and a computer is shown by comparing FIG. 1A (image) with FIG. 1B data (as pixels) contained in the computer from the image of FIG. 1B, which data in FIG. 1B is segmented. An example computer vision task would be to segment the image into different meaningful parts, such as by identifying all pixels that correspond to the iris, the pupil and the sclera. This is accomplished by using machine learning, and preferably deep learning principles. Deep learning is a type of machine learning methods that string together multiple connected layers of data processing. These different layers of data process learn different levels of representations corresponding to different levels of abstraction. In this manner there is a set of methods that automatically detect patterns in the data, e.g., information about an image of the eye, and the uncovered patterns to predict future data or make other decisions about the characteristics of the eye, under uncertainty.

Turning to FIG. 2, there is illustrated an example of a layer of data processing, which combines weighted sums with nonlinear functions. This may also be referred to as a neural network approach. Specifically, in the embodiment of FIG. 2, the layer of data processing produces weighted sums of inputs passed through a nonlinear function f which defines the value of each “neuron” in “hidden” layer h (can have multiple “such” hidden layers before output layer o). Following all layers of the network, a final probabilistic output is produced (for example, by a softmax classifier), from which a loss function can be computed. Gradients of the loss function with respect to each individual weight of each layer determine how the weights should be modified to obtain a more favorable loss function value when the same input is processed again.

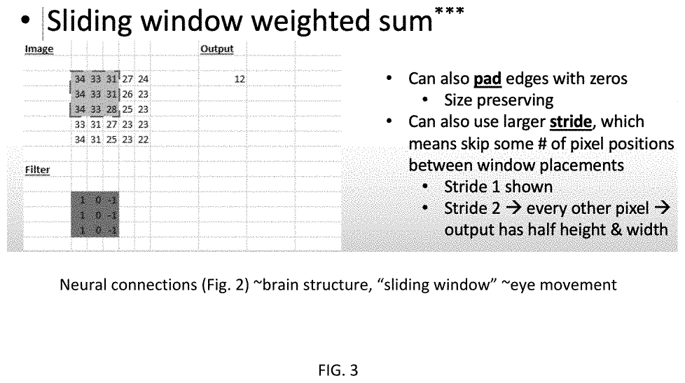

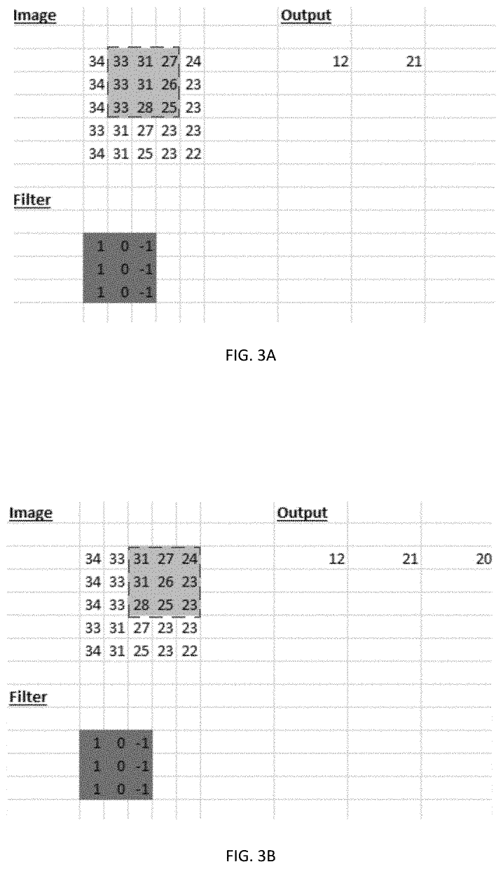

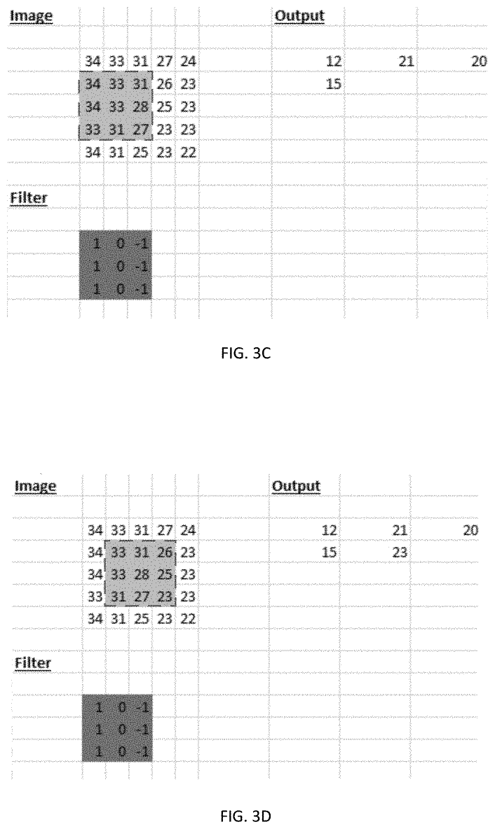

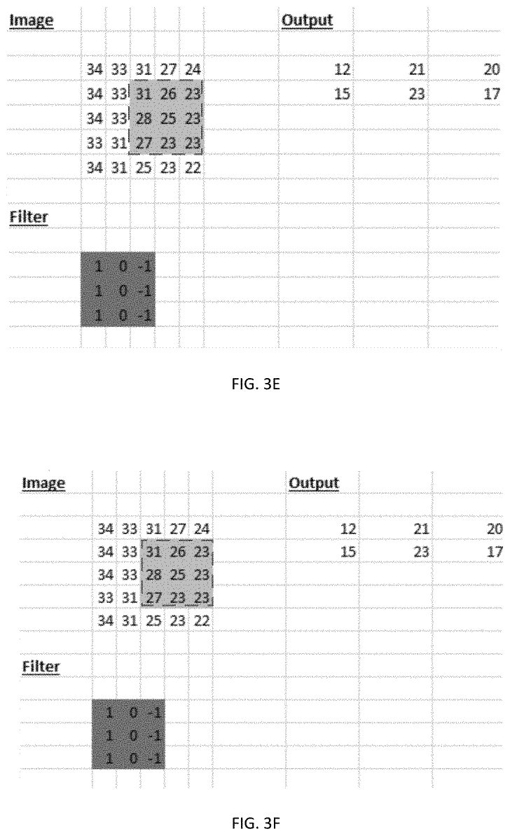

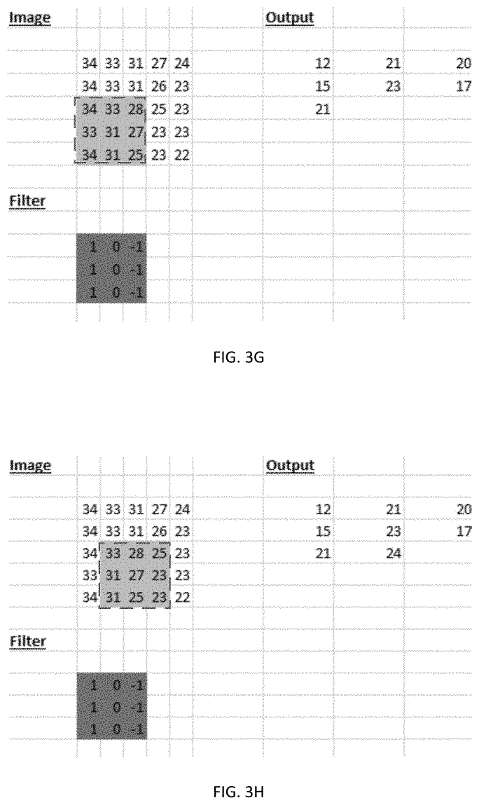

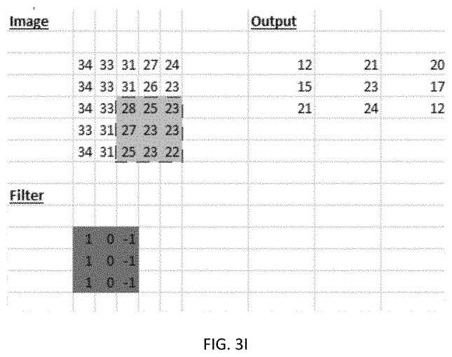

Turning to FIG. 3, and FIGS. 3A-3I there is illustrated an example of a layer of data processing, which is a convolution approach. In this approach the sliding window, the box (dashed box) identifies the section of the image (e.g., pixels) that are processed, this box moves to different parts of the image (as shown in FIGS. 3A to 3I) providing an output for each position of the window. This approach uses a weighted sum.

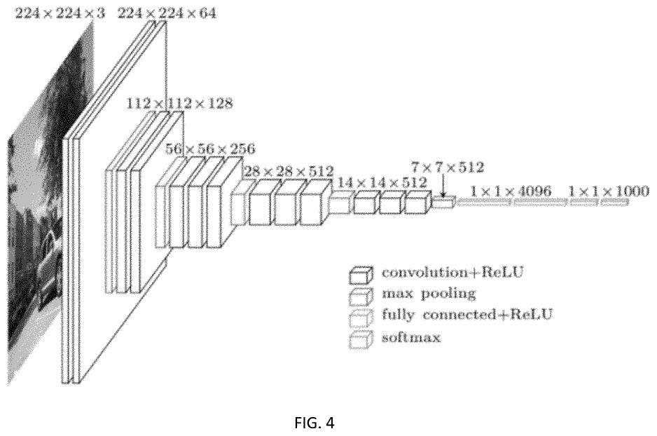

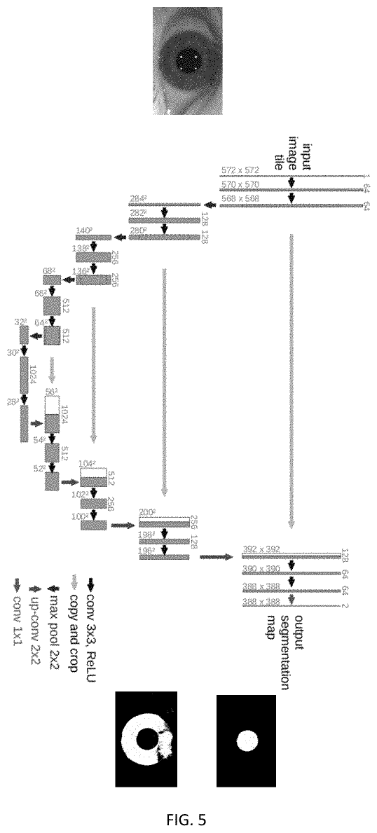

Turning to FIG. 4 there is an illustration of complete deep learning model, which is a convolutional neural network (CNN), which combines convolution layers with weighted sum layers and nonlinear functions. Any number of additional operations can be incorporated to a CNN, such as: max pooling, rectified linear unit (ReLU); batch normalization, dropout, and shortcut routing, among others. Max pooling is similar to convolution, but takes max value at each position. ReLU drops all negative output, e.g., it is set to zero, and exhibits nonlinearity.

Embodiments of a deep learning model have, and utilize, one or more of these layers. These different layers of data processing can be used individually, they can be used collectively, in parallel, serially, and in combinations and variations of these. Preferably, in a device incorporating such a deep learning model, various computationally intensive operations within the layers are run in parallel. In a preferred embodiment the hardware for the device is a GPU having thousands of computation cores and is thus highly parallelizable.

Example GPUs include the Nvidia RTX 3090, Nvidia RTX 3060, and the Nvidia RTX 2080. An alternative approach would be to utilize cloud computing to a specifically configured server containing the deep learning model. This could be configured, for example, with Amazon Web Services.

Information hierarchy can be viewed as a way in which the different layers and processes are used in conjunction with other layers and processes. FIG. 5 shows an illustration of a U-Net approach to information hierarchy, which balances the processing load across different information scales with a goal of producing an output at the same scale as the input.

The deep learning models need to be trained to achieve the optimum quality (e.g., accuracy) of the determined characteristics as necessary to achieve a given task.

In an embodiment of training a CNN device (e.g., a device having as computer with a program having a CNN having one or more layers of processes), the chain rule of calculus allows each individual weight of each filter to be modified in the direction of decreasing loss. Optimal efficiency is obtained by utilizing backpropagation, which traverses the layers in reverse order and leverages formulas for updating the weights of the current layer based on the already-computed weight update for the adjacent layer.

Iris registration devices for determining derived data and information about the position of the eye, including determining iris movement, and cyclotorsion of the eye, to enable a therapeutic laser beam delivery pattern to be accurately delivered to the eye, are taught and disclosed in US patent publication number 2015/0105759, the entire disclosure of which is incorporated herein by reference. Generally, images (optical images) travel back from the structure of the eye, the PID, or other structures, to the device or system along an optical path, e.g., along an image path, which can be through free space, optical components (lens, mirrors, fibers etc.) and both. In an embodiment of the present inventions, the processes and methods of determining iris registration, iris movement, cyclotorsion and combinations and variations of these are improved in quality (e.g., accuracy) using embodiments of the present deep learning methods to further process the raw data, derived information or both, to provide determined characteristics about the iris, iris movement and cyclotorsion.

A machine learning model may also use modeled data, observed historic data, derived historic data, and molded historic data, and combinations of these and other data and information sources, to provide a determined characteristic.

### Patient Interface—Generally

A further component of embodiments of these systems can be the laser patient interface or PID. It is noted that all or some of the PID is typically not a part of the system, but is preferably a single use device, (e.g., a disposable) that is added to the system for each patient prior to, or as set up for, a laser procedure. In embodiments, this interface provides that the x, y, z position between the natural lens and the laser remains fixed during the procedure, which includes both the measurement steps of determining the x y z position and the delivery step of delivering the laser to the lens in a shot pattern. The interface device may contain an optically transparent applanator. One example of this interface is a suction ring applanator that can be circular or elliptical that is fixed against the outer surface of the eye and is then positioned against the laser optical housing, thus fixing the distance between the laser, the eye and the natural lens. Reference marks for the 3-dimensional viewing and measuring apparatus may also be placed on this applanator. Moreover, the interface between the lower surface of the applanator and the cornea may be observable and such observation may function as a reference. A further example of a laser patient interface is a device having a lower ring, which has suction capability for affixing the interface to the eye. The interface further has a flat bottom, which presses against the eye flattening the eye's shape. This flat bottom is constructed of material that transmits the laser beam and also preferably, although not necessarily, transmits optical images of the eye within the visible light spectrum. The upper ring has a structure for engaging with the housing for the laser optics, some structure that is of known distance from the laser along the path of the laser beam and fixed with respect to the laser, and combinations and variations of these. Examples of patient interfaces devices, and system to engage the PID with the eye, and systems to engage the PID with the laser system, are disclosed and taught in US Patent Application Publication Nos. 2011/0190739, 2017/0290703, 2010/0022994, 2011/0022035 and 2015/0088175, the entire disclosures of each of which are incorporated herein by reference.

During testing and calibration of the laser system, a laser beam, imaging and preferably for calibration of the laser system after training of the augmented reality device, the therapeutic laser beam can be transmitted through the window of the PID.

### Ultrasound/Phacoemulsification—Generally

Any ultrasonic generator, e.g., ultrasonic driver, horn or other device to create ultrasonic energy, that is configured to provide ultrasonic energy that is useful, safe and effective for treating the eye, its structures and adjacent tissues, and conditions thereof, can be used to provide the ultrasonic energy for the present systems. In particular, some or all of the comports of any, preferably approved by a medical device regulatory body, phacoemulsification system can be used, or reconfigured for use, in embodiments of the present systems.

Generally, in embodiments of the present integrated systems, and the methods they can perform, phacoemulsification includes making a corneal incision, scleral incision (and combinations and variations of these) preferably with the therapeutic laser beam, and the insertion of a phacoemulsification handpiece, which is typically comprised of a needle that is ultrasonically driven, in order to, for example, emulsify, i.e., to liquefy, the natural crystalline lens, break a cataract into small pieces, and combinations and variations of these. Preferably, this ultrasonic procedure is performed on a lens, lens material, cataractous material, that has been cut, sectioned, softened and combinations and variations of these, by the laser beam. The emulsified pieces may subsequently be removed using the same handpiece or another handpiece. The surgeon may then insert implants, e.g., interocular lens, IOLs, in the eye through the incision,

In embodiments of the present systems, the phacoemulsification handpiece is generally coupled to an irrigation source and an aspiration pump. The aspiration pump is located a housing in the system. The handpiece includes a distal tip for insertion within the anterior chamber of the patient's eye that emits the ultrasonic energy, or vibrates at ultrasonic frequencies, to cut, to emulsify, and combinations and variations of these, the crystalline or natural lens. The handpiece further includes an irrigation port proximal to the distal tip, which is coupled to an irrigation source via an irrigation line, and an aspiration port at the distal tip, which is coupled to an aspiration pump via an aspiration line, Fluid from the irrigation source, which is typically an elevated bottle of saline solution, is irrigated into the eye via the irrigation line and the irrigation port, and the irrigation fluid and emulsified crystalline lens material are aspirated from the eye by the aspiration pump via the aspiration port and the aspiration line.

Phacoemulsification components, e.g., subassemblies, of embodiments of the present systems typically include a control system, e.g., a programmable microprocessor, a console with operator-selected presets for controlling, which in embodiments is the system monitor, for example, aspiration rate, vacuum and ultrasonic power levels. The phacoemulsification handpiece may be interconnected with the system by an electric cable for powering and controlling the piezoelectric transducer that provides the emulsification. Tubing provides the irrigation fluid to the eye, and enables withdrawal of aspiration fluid from an eye, through the handpiece under the control of the console.

The phacoemulsification ultrasound probe delivers energy into the eye that is used to break up the remaining cataractous lens material after laser fragmentation or cutting, to facilitate emulsification and aspiration of the remaining pieces. The phacoemulsification ultrasound probe delivers energy into the eye that is used to break up a cataract where no laser fragmentation of the cataract has been performed, for example where the surgeon elects to perform a laser capsulotomy and laser incision for insertion of the phacoemulsification probe, but does not use the laser to fragment the lens or cataract. The phacoemulsification ultrasound probe delivers energy into the eye that is used to break up the cataract accomplishes this by vibrating at a fixed frequency when the control switch, e.g., a foot pedal is depressed to predetermined position, the same control switch can control the firing or delivery of the therapeutic laser beam and pattern, other types of control switches, buttons, triggers, audio, etc. can be used. In embodiments, to increase the amount of ultrasound power, the machine increases the stroke length of the probe.

In embodiments, the phaco control system, alone or in combination with the system control system can perform various active monitoring and control functions such as: monitoring intraocular pressure (IOP) and can adjust the function of the system to maintain the IOP at the desired pressure; monitoring and controlling vacuum levels; optimizing power settings; and predicting pressure changes and proactively responding to occlusion breaks. The sensors to provide this information and data for the monitoring and autonomous control of the phaco system operating parameters can be in the system, on the various pumps and devices, based upon current or other electrical load, or in or on the hand piece.

An example of the performance features, and components that can be used, in the present phaco-laser systems is the Alcon CENTURION® Vision System and the Alco ACTIVE SENTRY® Handpiece and INTREPID® Hybrid Tip. In embodiments, the phaco handpiece can have a built-in fluidics pressure sensor that detects pressure in real time and communicates with the systems control system, the phocosystem control system or both. An example of the various features, and components that can be used, in the present phaco-laser systems is the AMO WHITESTAR SIGNATURE® PRO Phacoemulsification System.

Features, methods of using, and components of, phaco systems and subsystems are disclosed and taught in U.S. Pat. Nos. 8,020,565, 9,549,850, 9,549,851, 9,849,030, 9,877,865, 9,931,447, 9,937,077, 10,258,505, 10,314,953, 10,111,990, and US Published Patent Applications Nos. 2019/0133824, 2019/0021906, 2019/0099526, 2017/0266046, and 2017/0112668, he entire disclosures of each of which are incorporated herein by reference.

### EXAMPLES

The following examples are provided to illustrate various embodiments of systems, components of the systems, processes, compositions, applications and materials of the present inventions. These examples are for illustrative purposes, may be prophetic, and should not be viewed as limiting, and do not otherwise limit, the scope of the present inventions.

### Example 1

In an embodiment of an augmented reality device and processes, the device has programs configured as or providing a CNN. The CNN has many processing layers (3, 4, 5, 7 or more) with filters (1, 2, 3, 4 or more) in each layer. Each of the filters processes input using a sliding-window weighted sum approach (e.g., generally of the type show in FIGS. 3 to 3I). The CNN devices of this Example can be trained with a stochastic gradient descent (SGD) using a set of labeled images. The CNN is applied to image segmentation. In this manner the CNN is trained to classify all pixels in each image to match the provided hand-labeling of the training images.

### Example 2

This example presents results from a deep learning methodology to identify the pupil, limbus, and eyelid boundaries in images from a variety of commercially available topographers with a single unified software approach.

A sample of six hundred and four de-identified grayscale infrared iris images (each belonging to a different eye) were obtained from a variety of commercially available topographers. All images had the boundaries delineating the pupil and visible iris annotated by hand using software tools. A custom pupil warping method was utilized to produce augmented copies of each image with the iris “stretched” to varying pupil shapes and sizes. A deep convolutional neural network based on the U-Net architecture was trained to label pixels from these images as belonging to the pupil, iris, or neither. An active contours algorithm was developed to post-process the U-Net output into geometric curves and infer eyelid interference based on the shape of the iris contour. Performance was evaluated through two-fold cross-validation based on geometric center accuracy and dice coefficient (F1 score) of the final curve-bounded regions.

The pupil and limbus were identified in 603 out of 604 cases for a success rate of about 99.8%. The pupil center error—defined as the Euclidean distance between the center-of-mass of the hand-labeled pupil region and the center of the best-fit ellipse to the identified pupil curve—was below 5 pixels in 98.7% of cases, below 10 pixels in 99.5% of cases, and below 15 pixels in 99.7% of cases (pixel scaling varies from around 0.03 to 0.045 mm per pixel). The similarly defined limbus center error was below 5 pixels in 69.2% of cases, below 10 pixels in 95.0% of cases, and below 15 pixels in 99.8% of cases. Dice coefficients measured above 0.95 in 95.6% of cases for the pupil, 98.2% for the composite iris region (including the pupil area), and 92.0% for the exclusive iris region (excluding the pupil area).

This Example, among other things, demonstrates that an embodiment of a deep learning technology is capable of segmenting iris images from a wide variety of diagnostic devices under a unified software approach, with a standardized image resizing scheme serving as the only device-specific operation. This technological advance has significant implications for device interoperability, as it can enable device software to handle images from additional input devices with little or no change.

### Example 3

In an embodiment a computer vision device leveraging deep learning models is constructed, configured and operates as follows. This device has programs based upon C++ language and applications. (It being understood that other programing languages and applications may be used.) This device is fully integrable and preferably is fully integrated with, at least a, GUI. The deep learning model is trained, such as using the training method of EXAMPLE 4, and then transferred to the device.

### Example 4

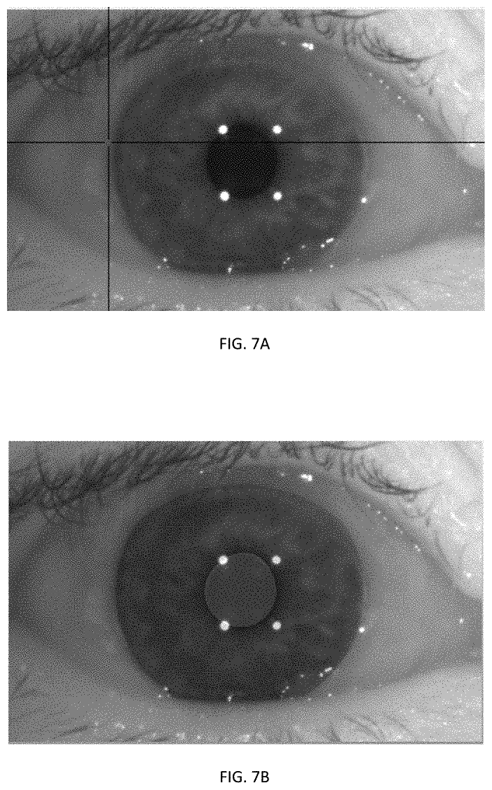

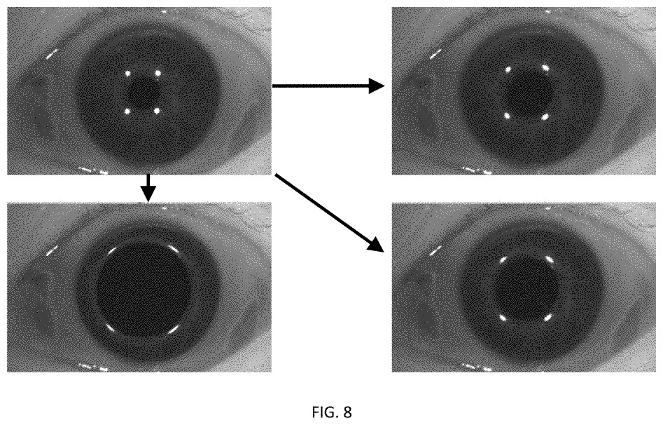

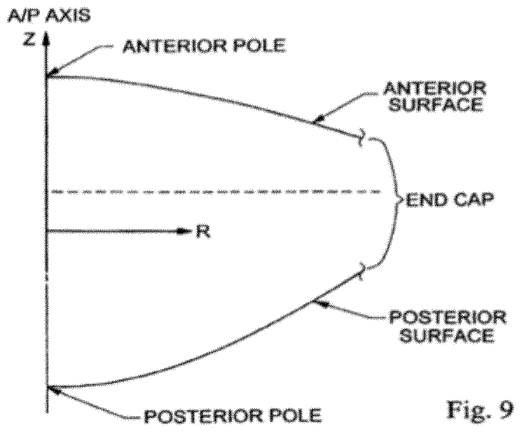

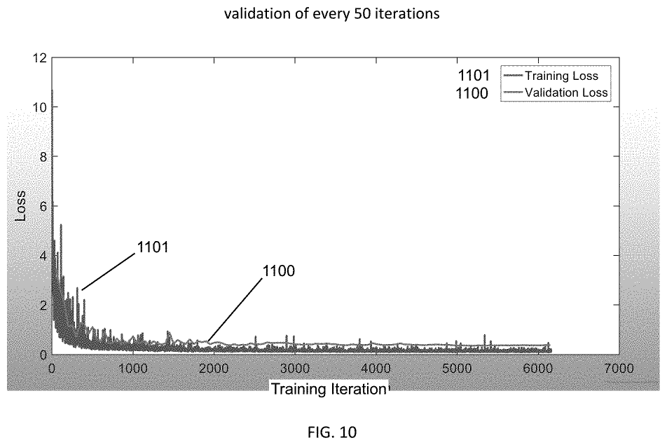

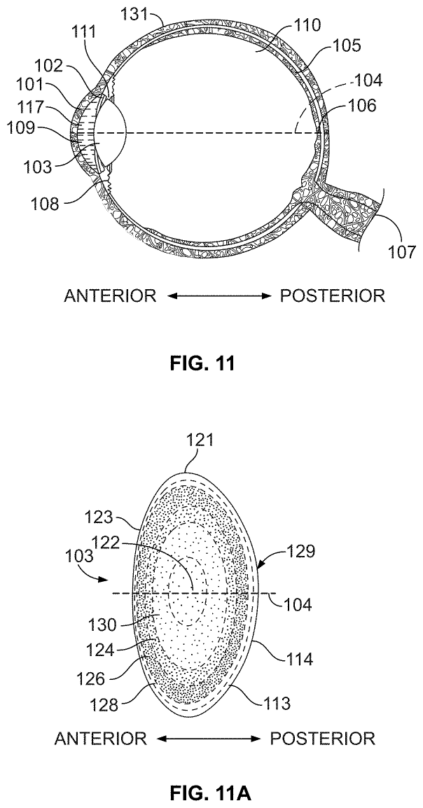

An embodiment of the devices and processes of the types disclosed in subheading “Providing Determined Characteristics—Generally”, of the type disclose in any of the other examples, and combinations and variations of these, is trained as follows.
Create a set of labeled images organize training set & validation set (See, e.g., FIGS. 7A and 7B)
(Optional) perform data augmentation on training (not validated) images
Set CNN architecture/params, start training
Observe validation set performance; revisit earlier steps as needed to improve
Trained CNN now available for usage on never-before-seen data; expected performance—that of validation set
Option—use cross validation for a more robust estimate of final performance
   2-way: split data into two halves, train on 1st half, test on 2nd half, then switch, compute average results, finally train on entire set
   Leave-one-out: only test on 1 image at a time
In embodiments, problem-specific data augmentation can be done instead of or in addition to more standardized techniques (e.g. flipping, stretching, adding noise).
   e.g., pupil warping in Matlab
   See FIG. 8
Example training and validation losses are shown in FIG. 10 (training loss shown as area 1101 and validation loss shown as line 1100)
Can also inspect validation image outputs over time
Monitoring of validation set guards against accidental over fitting.

### Example 5

An Embodiment of the devices and processes of the types disclosed in subheading “Providing Determined Characteristics—Generally”, of the type disclosed in any of the other examples, and combinations and variations of these is trained using a stochastic gradient descent (SGD), In this embodiment the gradient descent is a basic “hill climbing”. By stochastic it is understood at each iteration, there is used a random or (arbitrarily selected) approximation to a given loss function L rather than an exact value. For N training images, the exact loss value is L averaged over all N images. In the SGD approach n is used instead N, where n<N. The use of n, in place of N, may be referred to as a minibatch approach, with the batch size of n. This preferred embodiment avoids or minimizes the risk of the process getting stuck in local minima, and also, requires less memory.

### Example 6

An embodiment of the devices and processes of the types disclosed in subheading “Providing Determined Characteristics—Generally”, of the type disclose in any of the other examples, and combinations and variations of these has one or more of the additional processing tools and methods.

Regularization, which enforces certain mathematical properties on the weights in effort to prevent overfitting, which is effectively a “memorization” of the training data which struggles to generalize to other images. One example of regularization would be to penalize large weights.

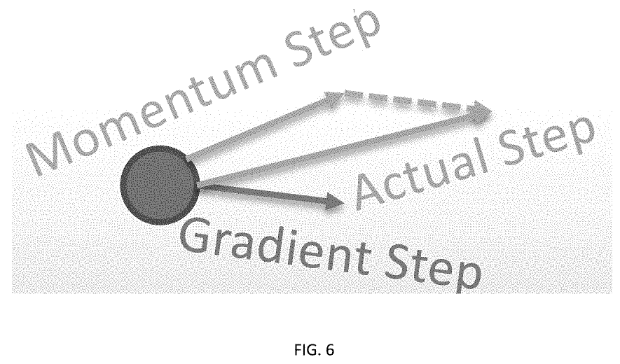

Momentum, which factors a previous weight update into the current weight update is illustrated in FIG. 6. Particularly when using a small batch size, momentum helps to preserve information gained from previous training iterations with each new step.

Data augmentation, which adds artificially generated images, such as from, a graphics generator or CAD system, modeled data or information. The system may also use historic observed data or information. In a preferred embodiment these training images are labeled. These artificially generated images can be scaled, rotated, noisy wrapped, and have variations in the level of quality, and consistency, etc. The use of data augmentation, among other things, produces a trained network that better generalizes to images outside of the original training set by forcing it to be capable of handling transformed versions of the training images.

### Example 7

In an embodiment a computer vision device and processes (CVD) of the types disclosed in subheading “Providing Determined Characteristics—Generally”, of the type disclosed in any of the other examples, and combinations and variations of these is a part of a medical device system. The CVD can be an integral part of the medical device, it can be a local separate device and connected by a network (e.g., hard wired, LAN or WIFI). The CVD can be in control communication with a laser eye surgery system, and in this manner provide image information and data, including information obtained through a deep learning model to that system. The CVD can be in control communication with a femto second laser eye surgery system, and in this manner provide image information and data, including information obtained through a deep learning model to that system. The CVD can be in control communication with an integrated laser-phacoemulsification eye surgery system, and in this manner provide image information and data, including information obtained through a deep learning model. The CVD can be in control communication with an integrated femtosecond laser—phacoemulsification eye surgery system, and in this manner provide image information and data, including information obtained through a deep learning model to that system.

### Example 8

Embodiments of these integrated ophthalmic systems, such as an embodiment of an integrated deep learning characterization phaco-laser system are configured to, and capable of addressing various conditions of the eye, and performing various procedures on the eye, including for example: capsulotomies; custom shaped non-circular and non-elliptical capsulotomies; lens cutting, fragmentation, sectioning and removal; cataract cutting, fragmentation, separation and removal; emulsification of lens and cataract tissue; corneal cutting and incisions; creation of corneal flaps and pockets; making limbal relaxing incisions; addressing and correcting refractive errors (natural and induced); removal of residual cortical material; removal of lens epithelial cells; vitreous aspiration and cutting associated with anterior vitrectomy; addressing bipolar coagulation; and intraocular lens injection.

### Example 9

Embodiments of these integrated ophthalmic systems, such as an embodiment of an integrated deep learning characterization computer vision device-phaco-laser system are configured to, and capable of addressing various conditions of the eye, and performing various procedures including keratoplasty, radial keratotomy (RK), astigmatic keratotomy (AK) and limbal relaxing incisions (LRI), and combinations and variations of these. These incisions can be made by the laser prior to or after the phaco procedure and lens implantation. RK would include, micro-RK, micro-RK/AK and traditional RK, while micro-RKs are preferred. For micro-RK the radial incisions can be used in optical zones that are preferably larger about 5.00 mm and larger, however smaller zones are contemplated. The length of the incisions is typically about 2.50 mm. The typical parameters for the incisions are set forth in Table III. Typically, 1, 2, 3, or more incisions are made in the cornea in a micro-RK procedure.

TABLE III

| Radial Incision | | Preferred | Default | |
|---|---|---|---|---|
| Parameter | Range | Value | Value | Units |
| Optical Zone | 4.00-6.25 | 5.00 | 5.00 | mm |
| Min. Clearance to AK | 0.00-0.50 | 0.20 | 0.20 | mm |
| Desired Radial Length | 0.50-2.50 | 2.00 | 2.00 | mm |
| Min. Residual Stroma | 100-300 | 150 | 150 | μm |
| Depth | 20-90 | 80 | 80 | % |
| Entrance Overcut (+) | −0.20-+0.20 | 0.06 | 0.06 | mm |

### Example 10

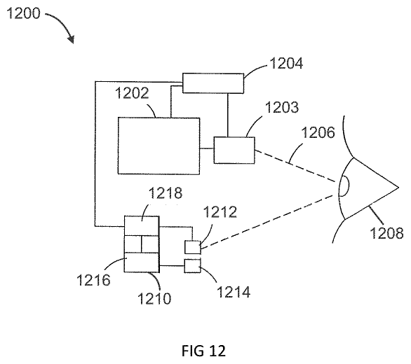

Turning to FIG. 12 there is shown a generalized schematic of a laser threptic system 1200. Such systems can be stand-alone ophthalmic therapeutic systems, or can be an integral part of a laser-phacoemulsification system. In a preferred embodiment, such systems are a laser-phacoemulsification systems (i.e., an integrated system having both a therapeutic laser and a phacoemulsification system), such as disclosed in US Pat. Publ. No. 2021/0259880, the entire disclosure of which is incorporated herein by reference.

Thus, and in general, the therapeutic laser system 1200 includes a laser source 1202, laser optics 1203, and a laser control system 1204 in communication thereto. The laser source 1202 generates a therapeutic laser beam 1206 that is directed to an eye 1208 of a patient via optics 1203. The laser beam 1206 is used to perform a variety of medical procedures on the eye 1208, such as capsulotomies, lens fragmentation, and corneal incisions, etc. The control system 1204 via its communication with the optics 1203 and the laser source 1202 controls a number of parameters of the laser beam, such as direction, pulse width, and pulse rate. Examples of a possible laser source 1202, optics 1203, and laser control system 1204 are disclosed in US Pat. Publ. No. 2021/0259880 and U.S. Pat. Nos. 8,262,646 and 8,465,478, the entire contents of each of which are incorporated herein by reference.

In communication with the laser source 1202 and laser control system 1204 is CVD 1210. The CVD 1210 has a processor 1218 and a memory 1216. As shown in this example, the CVD 1210 may also have associated with it a light source 1212 that illuminates the eye 1208, as well as, one or more detectors or cameras 1214 that receive light reflected off the eye 1208 and generate images of the eye 1208. The processor 1218 executes instructions stored in the memory 1216 so that a process using an algorithm is performed to analyze images of the eye and provide determined characteristics of the eye.

The CVD 1210 is of the types disclosed in subheading “Providing Determined Characteristics—Generally”, of the type disclosed in any of the other examples, and combinations and variations of these.

It is further understood that the CVD can be an integral part of, or contained within, the control system. The CVD may also be in the cloud, or remote from the laser system and in control communication with that system.

### Example 10A

Using the system of Example 10, one image of the eye 1208 is a pre-treatment image in that it is taken prior to the patient's eye 1208 being subjected to the therapeutic laser beam 1206. A second image of the eye 1208 is a treatment image and is taken substantially at the time the eye 1208 is treated by the therapeutic laser beam 106. The pretreatment and treatment images are stored in a recording medium, such as a memory 1216, and are processed in a processor 1218, which is in communication with the control system 1204, memory 1216 and light source 1212. The CVD then determines the cyclotorsion of the eye from the pre-treatment image and the treatment image and provides this information to the control system 1204, which adjust the laser deliver pattern for the therapeutic laser to compensate for, or otherwise address, the cyclotorsion of the eye.

### Example 10B

The process of Example 10A and thus the system of Example 10, determines iris movement, and thus cyclotorsion of the eye, to enable a therapeutic laser beam delivery pattern to be accurately delivered to the eye, by looking globally at the iris, and thus, eliminating the need for markers or specific reference points on the iris.

The system can also have processes, i.e. the execution of algorithms, with the CVD, that eliminate interference from eye lids.

### Example 11

A CVD (of the types disclosed in subheading “Providing Determined Characteristics—Generally”, of the type disclosed in any of the other examples, and combinations and variations of these) is configured to use of different imaging systems and differences in pupil dilation between preoperative and operative images cyclotorsion (and any other rotational alignment differences between sitting at the topographer and lying beneath the laser). The basic steps to the process are as follows.
1) Detect Pupil-Iris and Iris-Sclera boundaries in both images, as well as any eyelid interference.
2) Unwrap the iris and apply a Difference of Gaussians (DOG) Filter) to both unwrapped images.
3) Convert the unwrapped images from pixel representation to feature representation, where each pixel gives rise to one feature vector.
4) Measure global correlation strength between feature maps for each possible angle of cyclotorsion.
5) Repeat portions of steps 2-4 as required to optimize the unwrapping center of the laser image, so as to maximize the global maximum of the correlation function computed in step 4.
6) Take the angle that gives the strongest correlation, and thereby obtaining a determined characteristic, and rotate the coordinate system for the therapeutic laser delivery pattern accordingly.

### Example 12

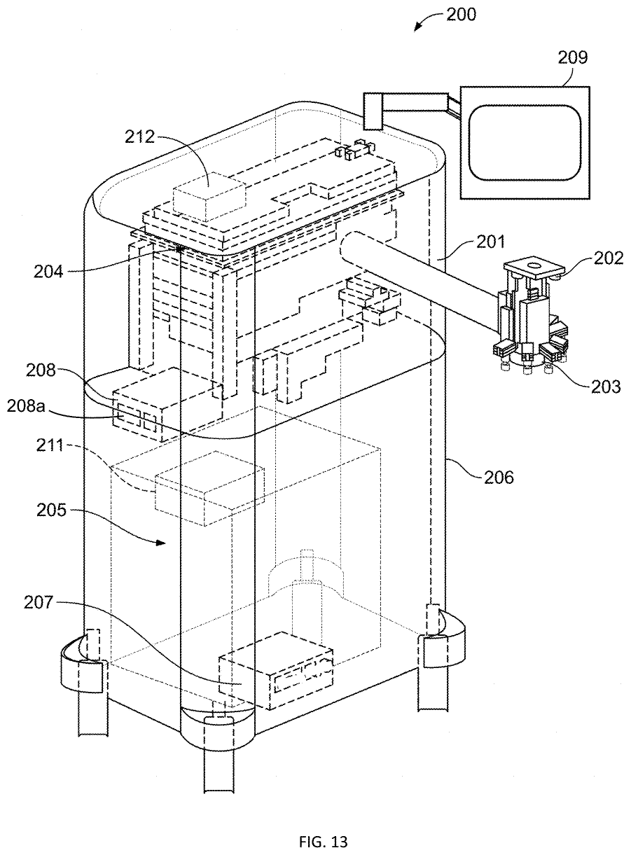

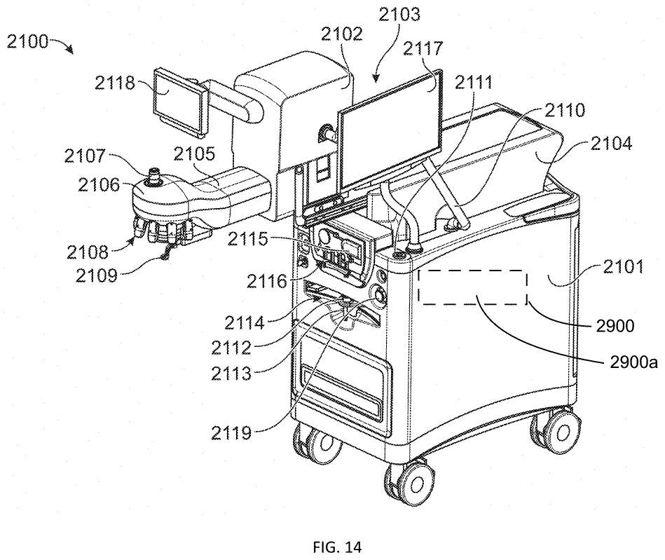

Turning to FIG. 13 there is shown a perspective, partial cutaway view of an embodiment of a femto-phaco laser system 200. The system 200 has a laser subsystem 204 and a phacoemulsification subsystem 205 that are contained within a common housing 206. The laser subsystem 204 includes a therapeutic laser beam source, and in embodiments a slow pulse duration and long pulse duration therapeutic laser beam source. The laser subsystem 204 includes a laser that defines a therapeutic laser beam path that the therapeutic laser beam travels along and optical components that are positioned or located along the laser beam path. These components would include z-direction focusing optics, and an x-y scanner.

The system 200 has an arm 201 that houses the therapeutic laser beam path, as well as other optical paths. In embodiments the arm 201 also houses or carries control and power cables for the imaging and position apparatus 203, and the docking assembly (not shown in this figure). The arm 201 has at its proximal end a therapeutic laser beam delivery head 202. The laser delivery head 202 has a position and shape deterring apparatus, which can be an OCT system, or the Scheimpflug systems of the present examples, and combinations and variations of these and other shape and position determining apparatus. The laser delivery head has a docking and positioning system that in conjunction with a PID docks to the patient's eye.

The system 200 has a common power supply 207 for the laser subsystem 204, and the phaco subsystem 205. The common power supply 207 provides all power for the entire system, eliminating the need for secondary power supplies, or sources of power. This permits the system to plugged into a single power supply in the operating room.

The system 200 has a common control system 208. The common control system 208 has a controller operating control software or operating instructions. The control system 208 has a CVD 208a (e.g., of the types disclosed in subheading “Providing Determined Characteristics—Generally”, of the type disclosed in any of the other examples, and combinations and variations of these is a part of a medical device system). In a preferred embodiment the common control system 208 is in control communication with one or more of and preferably all of: a control system and controller 212 in the laser subsystem; a control system and controller 211 in the phaco subsystem; in control communication with the operator interface 209; in control communication with the emergency stop 210; and in communication with a network that is for example a patient medical record system, an accounting system, and combinations and variations of these configurations.

Typically, the docking system, and imaging and position determining apparatus are controlled by the laser subsystem control system. In embodiments they may in whole or in part be controlled directly by the common control system 208.

In an embodiment the laser control system and the phaco control system are partially, and can be fully integrated into a single common control system. Thus, in an embodiment only one control system, or a single control system is present in the femto-phaco system.

This CVD 208a is configured to perform and performs the processes of Example 10B and 11, among others.

### Example 13

Turning to FIG. 13, there is shown a various view and components of embodiment of a femto-phaco laser system 2100. The system 2100 has a laser subsystem and a phacoemulsification subsystem that are contained within a common housing. The laser subsystem includes a therapeutic laser beam source, and in embodiments a slow pulse duration and long pulse duration therapeutic laser beam source. The laser subsystem includes a laser that defines a therapeutic laser beam path that the therapeutic laser beam travels along and optical components that are positioned or located along the laser beam path. These components would include z-direction focusing optics, and an x-y scanner.

The femto-phaco system 2100 has a first housing 2101 and an optical assembly and scanner housing 2102, that is movably, mechanically and optically associated with the first housing 2101. The housing 2101 is part of an extendable/retractable assembly 2103 that provides horizontal movement. The assembly 2103 also includes base 2104, which is mechanically associated with housing 2101, and is not movable with respect to housing 2101.

The optical assembly and scanner housing 2102 has an arm 2105 that houses portions of the laser beam path and the optical paths, and thus provides for transmission of the laser beam and optical images. Mechanically and optically associate with the arm 2105 is a laser delivery and imaging head 2106. Vertical movement of the head 2016 is controlled by joy stick 2017 (and may also be done alone or in combination with the control system, and also through the GUI). The vertical movement can be accomplished by any of the various devices disclosed in this specification.

The head 2016 has a position and determining device 2108 and a patient interface device 2109.

A laser beam movable connector device, e.g., articulated hollow pipe 2110, transmits the laser beam along a laser beam path for the laser in housing 2101 to the optics and beam handling devices in housing 2102.

The system has an opening 2114 for holding and storing a phaco tray assembly 2112. The phaco tray assembly has a frame 2122 that holds a support tray 2121 and an engagement pin 2113.

The system 2100 has two openings 2111 (left side) and right side (not shown) for receiving and holding pin 2113 and thus the phaco tray assembly. In this manner the system 2100 is non-handed, such that it can be configured for use in identical manners on both the right and left had sides, and the tray and tray assembly is non-handed, as it can be used on both the left and right sides of the system.

The system 2100 has a phaco cassette 2115 and ports 2116 for connecting phaco related components (e.g., air, VIT, DIA, control cables, etc.).

The system 2100 has two monitors 2117, 2118 that are preferably graphic user interfaces (GUI) that display information and menus and take input, e.g., instructions from the operator of the system. The GUIs can display control and information menus.

The system 2100 has an emergency stop 2119.

In the embodiment of this example the position and determining device 2108 has 5 or 5 Scheimpflug camera assemblies. The head 2106 has a laser delivery opening (not shown). The laser beam path and the image path go through this opening. The opening can have a clear window, transmissive to the laser beam and images, it can also have a cap, iris or other closure device that closes the window when the laser is not being used, e.g., when the device is in a retracted position, or when the phaco system is being operated.

The PID 2109 is attached to the laser head 2106, and thus the laser system 2100, through PID locking and engagement device, which has a locking lever tab that is moveable between a locked position and an unlocked position.

The arm 2105 that houses the therapeutic laser beam path, as well as other optical paths. In embodiments the arm 2105 also houses or carries control and power cables for the imaging and position apparatus and the docking assembly (not shown in this figure).

The system 2100 has a common power supply for the laser subsystem, and the phaco subsystem. The common power supply provides all power for the entire system, eliminating the need for secondary power supplies, or sources of power. This permits the system to plugged into a single power supply in the operating room.

The system 2100 has a common control system 2900, that has a CVD 2900a, (e.g., of the types disclosed in subheading “Providing Determined Characteristics—Generally”, of the type disclosed in any of the other examples, and combinations and variations of these is a part of a medical device system). The common control systems also includes emergency stop button or switch 2119. The common control system has a controller operating control software or operating instructions. In a preferred embodiment the common control system is in control communication with one or more of and preferably all of: a control system and controller in the laser subsystem; a control system and controller in the phaco subsystem; in control communication with the operator interface; in control communication with the emergency stop 2119; and in communication with a network that is for example a patient medical record system, an accounting system, and combinations and variations of these configurations.

This CVD 2900ais configured to perform and performs the processes of Example 10B and 11, among others.

### Example 14

The systems of Examples 10, 12 and 13 adjust the delivery of an ophthalmic therapeutic laser beam pattern, in an ophthalmic therapeutic laser beam system, by obtaining raw image data from an iris of an eye; processing the raw image data in a deep learning means to thereby provide a determined characteristic of the eye; using the determined characteristic to adjust a delivery location for the therapeutic laser beam pattern. These adjustments can be automatic, programed to be automatic, and can be with or without user approval.

### Headings and Embodiments

It should be understood that the use of headings in this specification is for the purpose of clarity, reference, and is not limiting in any way. Thus, the processes compositions, and disclosures described under a heading should be read in context with the entirely of this specification, including the various examples. The use of headings in this specification should not limit the scope of protection afforded the present inventions.

It is noted that there is no requirement to provide or address the theory underlying the novel and groundbreaking processes, laser operations, and laser patterns, enhanced and improved vision, or other beneficial features and properties that are the subject of, or associated with, embodiments of the present inventions. Nevertheless, various theories are provided in this specification to further advance the art in this area. The theories put forth in this specification, and unless expressly stated otherwise, in no way limit, restrict or narrow the scope of protection to be afforded the claimed inventions. These theories many not be required or practiced to utilize the present inventions. It is further understood that the present inventions may lead to new, and heretofore unknown theories to explain the function-features of embodiments of the methods, laser patterns, laser operations, functions of the eye, devices and system of the present inventions; and such later developed theories shall not limit the scope of protection afforded the present inventions.

The various embodiments of devices, systems, laser shot patterns, activities, and operations set forth in this specification may be used with, in or by, various measuring, diagnostic, surgical and therapeutic laser systems, in addition to those embodiments of the Figures and disclosed in this specification. The various embodiments of devices, systems, laser shot patterns, activities, and operations set forth in this specification may be used with: other measuring, diagnostic, surgical and therapeutic systems that may be developed in the future: with existing measuring, diagnostic, surgical and therapeutic laser systems, which may be modified, in-part, based on the teachings of this specification; and with other types of measuring, diagnostic, surgical and therapeutic systems. Further, the various embodiments of devices, systems, laser shot patterns, activities, and operations set forth in this specification may be used with each other in different and various combinations. Thus, for example, the configurations provided in the various embodiments of this specification may be used with each other. For example, the components of an embodiment having A, A′ and B and the components of an embodiment having A″, C and D can be used with each other in various combination, e.g., A, C, D, and A, A″ C, D, and A′, D, etc., in accordance with the teaching of this specification. Thus, the scope of protection afforded the present inventions should not be limited to a particular embodiment, configuration or arrangement that is set forth in a particular embodiment, example, or in an embodiment in a particular Figure.

The inventions may be embodied in other forms than those specifically disclosed herein without departing from their spirit or essential characteristics. The described embodiments are to be considered in all respects only as illustrative and not restrictive.

1. An ophthalmic therapeutic laser system, comprising:
a. an assembly, the assembly comprising:
i. a therapeutic laser for providing a therapeutic laser beam along a laser beam path;
b. an arm attached to the assembly;
i. the arm having a distal end and a proximal end, wherein the distal end is attached to the assembly;
ii. wherein the proximal end has a laser delivery head;
iii. wherein the arm contains a portion of the laser beam delivery path; and,
c. a deep learning means for providing one or more of an image, data and information for a structure of an eye.

2. The laser system of claim 1, wherein the deep learning means provides targeting information for the direction, placement or both of a therapeutic laser beam shot pattern.

3. The laser system of claim 2, wherein the targeting information comprises a cyclotorsion of the eye based solely upon a retina of the eye.

4. The laser system of claim 3, wherein the system further comprises a phacoemulsification system for providing therapeutic ultrasonic energy to the eye.

5. The laser system of claim 4, wherein the phacoemulsification system in integrated with the laser system and shares, at least a part of, one or more of a common housing, a common control system, a common power source.

6. The laser system of claim 1, wherein the deep learning means comprises a computer vision device (CVD).

7. The laser system of claim 6, wherein the deep learning means comprises a computer vision device (CVD), wherein the CVD is based upon a convolutional neural network.

8. The laser system of claim 6, wherein the deep learning means comprises a computer vision device (CVD), wherein the CVD is trained by a convolutional neural network.

9. The laser system of claim 6, wherein the deep learning means comprises a computer vision device (CVD), wherein the CVD is based upon a U-Net approach to information.

10. The laser system of claim 6, wherein the deep learning means comprises a computer vision device (CVD), wherein the CVD is trained by a U-Net approach to information.

11. An ophthalmic therapeutic laser system, comprising:
a. an assembly, the assembly comprising:
i. a therapeutic laser for providing a therapeutic laser beam, in a laser beam pattern, along a laser beam path;
b. an arm attached to the assembly;
i. the arm having a distal end and a proximal end, wherein the distal end is attached to the assembly;
ii. wherein the proximal end has a laser delivery head;
iii. wherein the arm contains a portion of the laser beam delivery path; and,
c. a deep learning means for providing a determined characteristic about a structure of an eye.

12. The system of claim 11, wherein the means for providing determined characteristics is a computer vision device (CVD).

13. The laser system of claim 12, wherein the CVD is based upon a convolutional neural network.

14. The laser system of claim 12, wherein the CVD is trained by a convolutional neural network.

15. The laser system of claim 12, wherein the CVD is based upon a U-Net approach to information.

16. The laser system of claim 12, wherein the CVD is trained by a U-Net approach to information.

17. The laser system of claim 11, wherein the structure of the eye is the retina.

18. The laser system of claim 11, wherein the determined characteristics are not based upon an individual markers or specific reference points on the iris.

19. The laser system of claim 11, wherein the determined characteristic provides in part targeting information for the delivery of the laser beam pattern.

20. The laser system of claim 11, wherein the determined characteristic is the cyclotorsion of the eye.

21. The laser system of claim 11, wherein the determined characteristic is the cyclotorsion of an undilated pre-treatment eye and a dilated treatment eye.

22. The laser system of claim 11, wherein the determined characteristic is the cyclotorsion of an eye having different amounts of dilation between pre-treatment and treatment.

23. The laser system of claim 11, wherein the system further comprises a phacoemulsification system for providing therapeutic ultrasonic energy to the eye.

24. The laser system of claim 11, wherein the system further comprises a phacoemulsification system for providing therapeutic ultrasonic energy to the eye; wherein the phacoemulsification system in integrated with the laser system and shares, at least a part of, one or more of a common housing, a common control system, a common power source.

25. A method of adjusting the delivery of an ophthalmic therapeutic laser beam pattern, in an ophthalmic therapeutic laser beam system, the method comprising: obtaining raw image data from an iris of an eye; processing the raw image data in a deep learning means to thereby provide a determined characteristic of the eye; using the determined characteristic to adjust a delivery location for the therapeutic laser beam pattern.

26. The method of claim 25, wherein the laser beam system further comprises a phacoemulsification system.

27. The method of claim 26, wherein the determined characteristic is a cyclotorsion of the eye.

28. The method of claim 27, wherein the cyclotorsion of an undilated pre-treatment eye and a dilated treatment eye.

29. The method of claim 27, wherein the cyclotorsion of an eye having different amounts of dilation between pre-treatment and treatment.

30. The method of claim 25, wherein the determined characteristics are not based upon an individual markers or specific reference points on the iris.

31. The method of claim 25, wherein the deep learning means comprises a computer vision device (CVD).

32. The method of claim 25, wherein the deep learning means comprises a computer vision device (CVD), wherein the CVD is based upon a convolutional neural network.

33. The method of claim 25, wherein the deep learning means comprises a computer vision device (CVD), wherein the CVD is trained by a convolutional neural network.

34. The method of claim 25, wherein the deep learning means comprises a computer vision device (CVD), wherein the CVD is based upon a U-Net approach to information.

35. The method of claim 25, wherein the deep learning means comprises a computer vision device (CVD), wherein the CVD is trained by a U-Net approach to information.

36. The system of claim 1, wherein the system is configured to provide two therapeutic laser beams having different pulse durations.

37. The system of claim 1, wherein the therapeutic laser is a femto second laser; and wherein the system is configured to provide two therapeutic laser beams having different pulse durations; wherein both therapeutic laser beams are configured to ablate tissue, cut tissue, or both.

38. The system of claim 1, wherein the system comprises a surgical microscope; and the surgical microscope is integral with the system and configured to receive one or more of images, data, information from the laser system; wherein the surgical microscope is configured to display the received images, data or information, including images, data and information from the deep learning means during a laser procedure, a phacoemulsification producer, or both.

39. The system of claim 1, wherein the system comprises a 3D viewing system; and the 3D viewing system is integral with the system and configured to receive one or more of images, data, information from the laser system; wherein the 3D viewing system is configured to display the received images, data or information during a laser procedure, a phacoemulsification producer, or both.

40. The system of claim 1, wherein the system comprises a foot switch in control communication with one or more of the integration control system, the therapeutic laser control system, and the phacoemulsification control system.

41. The system of claim 1, wherein:
a. the integration control system, the therapeutic laser control system or both have a plurality of predetermined laser delivery patterns;
b. the integration control system, the phacoemulsification control system or both have, a plurality of predetermined phacoemulsification procedures; and,
c. the deep learning means is configured to determine information about a cataract in a lens of an eye, and the system is configured based upon that determined information to recommend, at least in part, a laser-phaco combined therapy based upon the determined information about the cataract; wherein the laser-phaco combined therapy comprises:
i. at least one of the plurality of predetermined laser delivery patterns; and,
ii. at least one of the plurality of predetermined phacoemulsification producers.

42. The system of claim 1, wherein the deep learning means provides iris registration information.

43. A method of using an integrated laser-phaco system comprising: a GUI; a therapeutic laser for providing a therapeutic laser beam along a laser beam delivery path, comprising a therapeutic laser control system; a phacoemulsification system for providing therapeutic ultrasonic energy, comprising a phacoemulsification system control system; an integration control system in control communication with the therapeutic laser control system, a deep learning means for providing an image, data and/or information for a structure of an eye, the phacoemulsification system and the GUI, to determine, provide or both, a laser-phaco combined therapy for a cataractous eye of a patient, the method comprising:
a. the system evaluating information about a cataract in a lens of the cataractous eye of the patient;
b. the system determining a recommended laser-phaco combined therapy, based at least in part, upon the determined information about the cataract; wherein the recommended laser-phaco combined therapy comprises a predetermined laser delivery pattern, and a predetermined phacoemulsification procedure;
c. the system displaying on the GUI the menu items relating to the recommended laser-phaco combined therapy;
d. the system receiving a selection of the recommended laser-phaco combined therapy for deliver to the lens of the eye of the patient.

44. A system comprising:
a. an ophthalmic device; and,
b. deep learning means for providing a determined characteristic of an eye.

45. The system of claim 44, wherein the determined characteristic is one or more of an image, data, targeting information for a structure of an eye.

46. The system of claim 45, wherein the structure of the eye is an iris.
